(19) United States
(12) Patent Application Publication
(10) Pub. No.: US 2014/0121540 A1
(43) Pub. Date: May 1, 2014

(54) SYSTEM AND METHOD FOR MONITORING THE HEALTH OF A USER

(71) Applicant: Aza Raskin, San Francisco, CA (US)

(72) Inventor: Aza Raskin, San Francisco, CA (US)

(73) Assignee: AliphCom, San Francisco, CA (US)

(21) Appl. No.: 13/890,143

(22) Filed: May 8, 2013

Related U.S. Application Data
(60) Provisional application No. 61/644,917, filed on May 9, 2012.

(51) Int. Cl. A61B 5/00; A61B 5/16; A61B 5/107; A61B 5/0205

(52) U.S. Cl. CPC A61B 5/0079; A61B 5/0013; A61B 5/0205; A61B 5/165; A61B 5/1079; A61B 5/742; A61B 5/681; A61B 5/0082; A61B 5/02416
USPC 600/479; 600/476

(57) ABSTRACT

Embodiments of the present application relate generally to electrical and electronic hardware, computer software, wired and wireless network communications, wearable, hand held, portable computing devices for facilitating communication of information, and the fields of healthcare and personal health. More specifically the present application relates generally to the field of personal health, and more specifically to new and useful systems and methods for monitoring the health of a user as applied to the field of healthcare and personal health. A system optically detects facial features of a user and analyzes the features along with weight information about the user to make one or more recommendations to the user that are related to the user's health. The weight information may be wirelessly transmitted to the system by a wirelessly-enabled scale (e.g., a bath scale), data capable strap band, wristband, wristwatch, digital watch, or wireless activity monitoring and reporting device.

### CROSS-REFERENCE TO RELATED APPLICATIONS

This application Claims the Benefit of and Priority to U.S. Provisional Patent Application Ser. No. 61/644,917, filed on May 9, 2012, having attorney docket number MSSV-P06-PRV, and titled “SYSTEM AND METHOD FOR MONITORING THE HEALTH OF A USER” which is hereby incorporated by reference in its entirety for all purposes.

This application is related to U.S. Provisional Application Ser. No. 61/641,672, filed on 2 May 2012, having attorney docket number MSSV-P04-PRV, and titled “METHOD FOR DETERMINING THE HEART RATE OF A SUBJECT”, which is hereby incorporated by reference in its entirety for all purposes.

### FIELD

This present application relates generally to the field of personal health, and more specifically to new and useful systems and methods for monitoring the health of a user applied to the field of healthcare and personal health.

### BACKGROUND

With many aspects of stress, diet, sleep, and exercise correlated with various health and wellness effects, the rate of individuals engaging with personal sensors to monitor personal health continues to increase. For example, health-related applications for smartphones and specialized wristbands for monitoring user health or sleep characteristics are becoming ubiquitous. However, these personal sensors, systems, and applications fail to monitor user health in a substantially holistic fashion and to make relevant short-term and long-term recommendations to users. The heart rate of an individual may be associated with a wide variety of characteristics of the individual, such as health, fitness, interests, activity level, awareness, mood, engagement, etc. Simple to highly-sophisticated methods for measuring heart rate currently exist, from finding a pulse and counting beats over a period of time to coupling a subject to an EKG machine. However, each of these methods requires contact with the individual, the former providing a significant distraction to the individual and the latter requiring expensive equipment.

Thus, there is a need in the fields of healthcare and personal health to create a new and useful methods, systems, and apparatus for monitoring the health of a user, including non-obtrusively detecting physiological characteristics of a user, such as a user's heart rate.

### BRIEF DESCRIPTION OF THE DRAWINGS

Various embodiments or examples (“examples”) of the present application are disclosed in the following detailed description and the accompanying drawings. The drawings are not necessarily to scale:

FIG. 1A depicts one example of a schematic representation of a system according to an embodiment of the present application;

FIG. 1B depicts another example of a schematic representation of one variation according to an embodiment of the present application;

FIG. 1C depicts a functional block diagram of one example of an implementation of a physiological characteristic determinator according to an embodiment of the present application;

FIG. 2 depicts an exemplary computer system according to an embodiment of the present application;

FIGS. 3A-3D depict graphical representations of outputs in accordance with a system or a method according to an embodiment of the present application;

FIG. 4A depicts a flowchart representation of a method according to an embodiment of the present application;

FIG. 4B depicts a flowchart representation of a variation of a method according to an embodiment of the present application;

FIG. 4C-6 depict various examples of flowcharts for determining physiological characteristics based on analysis of reflected light according to an embodiment of the present application;

FIG. 7 depicts an exemplary computing platform disposed in a computing device according to an embodiment of the present application; and

FIG. 8 depicts one example of a system including one or more wireless resources for determining the health of a user according to an embodiment of the present application.

### DETAILED DESCRIPTION

Various embodiments or examples may be implemented in numerous ways, including as a system, a process, an apparatus, a user interface, or a series of program instructions on a non-transitory computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying drawing FIGS. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

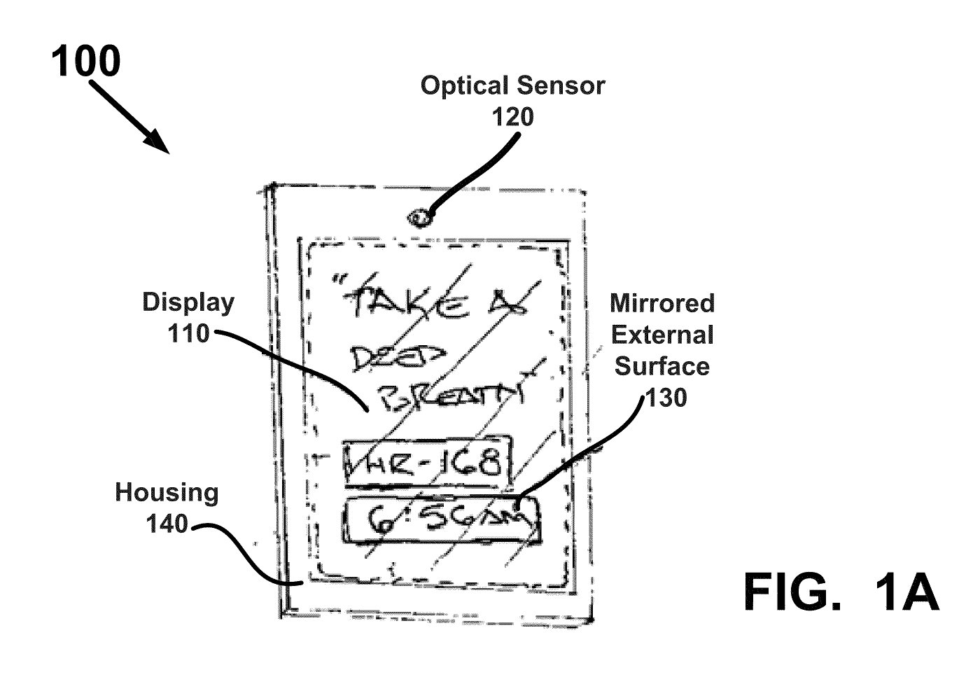

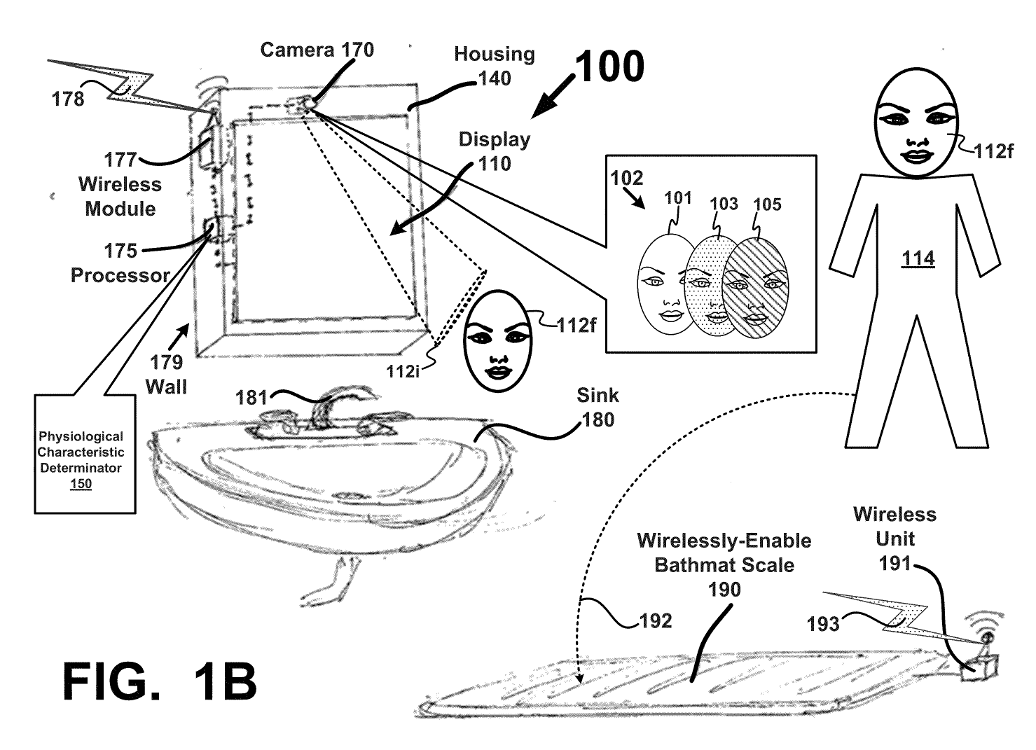

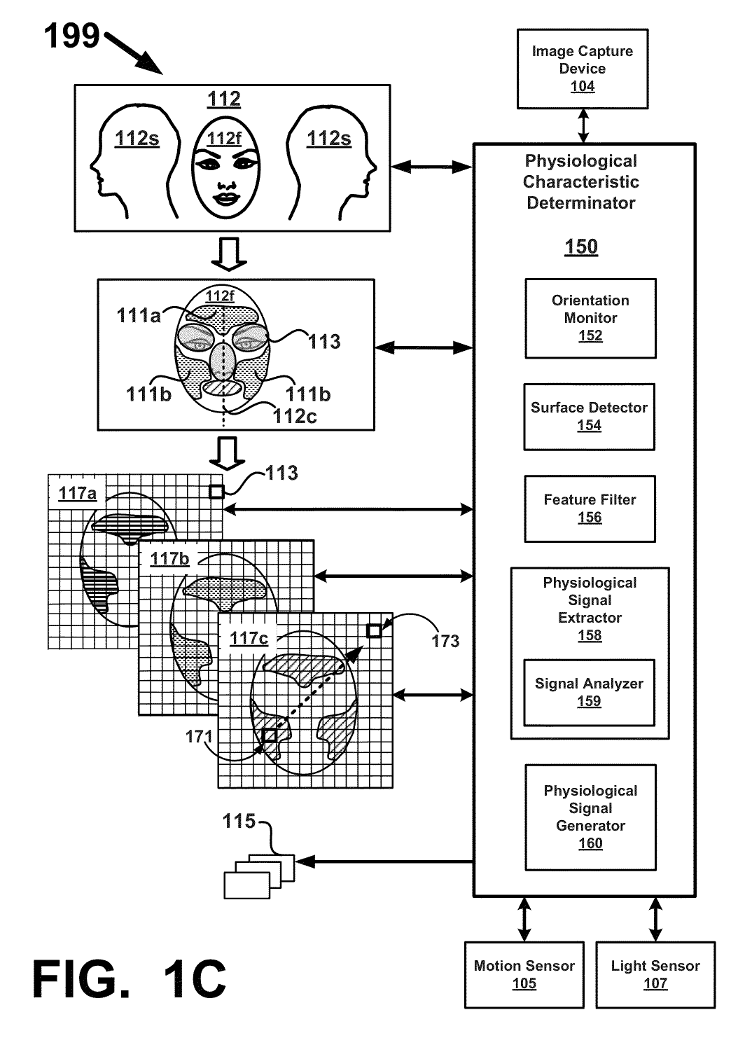

As depicted in FIGS. 1A and 1B, a system 100 for monitoring the health of a user 114 includes: a housing 140 configured for arrangement within a bathroom and including a mirrored external surface 130; an optical sensor 120 arranged within the housing 140 and configured to record an image 112i including the face 112f of a user 114; and a display 110 arranged within the housing 140 and adjacent the mirrored surface 130. The system 100 may additionally include a processor 175 that is configured to selectively generate a first recommendation for the user 114, based upon short-term data including a first current health indicator identified in the image of the user 114, and a second recommendation for the user 114, based upon the first current health indicator, a second current health indicator that is the weight of the user 114, and historic health indicators of the user 114. Housing 140 may be configured to be mounted to a surface such as a wall (e.g., wall 179) or other structure.

The system 100 preferably functions to deliver short-term recommendations to the user 114 based upon facial features extracted from an image of the user 114. The system 100 may further function to deliver long-term recommendations to the user 114 based upon facial features extracted from the image 112i of the user 114 and the weight of the user 114. The first current health indicator may be user heart rate, mood, stressor, exhaustion or sleep level, activity, or any other suitable health indicator. The current health indicator is preferably based upon any one or more of user heart rate, respiratory rate, temperature, posture, facial feature, facial muscle position, facial swelling, or other health-related metric or feature that is identifiable in the image 112i of the user 114 (e.g., image 112i of face 112f). The first current health indicator is preferably determined from analysis of the present or most-recent image of the user 114 taken by the optical sensor 120, and the first, short-term recommendation is preferably generated through manipulation of the first current health indicator. The first, short-term recommendation is preferably immediately relevant to the user 114 and includes a suggestion that the user 114 may implement substantially immediately. Historic user health-related metrics, features, and indicators are preferably aggregated with the first current health indicator and the second current health indicator, which is related to user weight, to generate the second, long-term recommendation. The second, long-term recommendation is preferably relevant to the user 114 at a later time or over a period of time, such as later in the day, the next day, or over the following week, month, etc., though the first and second recommendations may be subject to any other timing.

The system 100 is preferably configured for arrangement within a bathroom such that user biometric data (e.g., user facial features, heart rate, mood, weight, etc.) may be collected at regular times or intended actions of the user 114, such as every morning when the user 114 wakes and every evening when the user 114 brushes his teeth before bed. The system 100 may therefore be configured to mount to a wall adjacent a mirror or is configured to replace a bathroom mirror or vanity (e.g., on wall 179 and above sink 180 of FIG. 1B). Alternatively, the system 100 may be arranged on a bedside table, in an entry way in the home of the user 114, adjacent a television or computer monitor, over a kitchen sink, on a work desk, or in any other location or room the user 114 frequents or regularly occupies. In another variation of system 100, the system 100 functions are arranged over a crib, in a baby's room, or in a child's room as a baby or child monitor, wherein at least one the first and second recommendations are directed toward the parent of the user 114 who is a baby or child of the parent. In this variation, the system 100 may therefore function to monitor the health and wellness of a child, such as whether the child is becoming or is ill, is eating properly, is growing or developing as expected, or is sleeping well. However, the system 100 may be used in any other way and to monitor the health of any other type user and to provide the recommendations to the user 114 or any other representative thereof.

The system 100 preferably collects and analyzes the image 112i of the user 114 passively (i.e. without direct user prompt or intended input) such that a daily routine or other action of the user 114 is substantially uninterrupted while user biometric data is collected and manipulated to generate the recommendations. However, the system 100 may function in any other way and be arranged in any other suitable location.

The system 100 preferably includes a tablet computer or comparable electronic device including the display 110, a processor 175, the optical sensor 120 that is a camera 170, and a wireless communication module 177, all of which are contained within the housing 140 of the tablet or comparable device. Alternatively, the system 100 may be implemented as a smartphone, gaming console, television, laptop or desktop computer, or other suitable electronic device. In one variation of the system 100, the processor 175 analyzes the image 112i captured by the camera 170 and generates the recommendations. In another variation of the system 100, the processor 175 collaborates with a remote server to analyze the image 112i and generate the recommendations. In yet another variation of the system 100, the processor 175 handles transmission of the image 112i and/or user weight data, through the wireless communication module 177, to the remote server, wherein the remote server extracts the user biometric data from the image 112i, generates the recommendations, and transmits the recommendations back to the system 100. Furthermore, one or more components of the system 100 may be disparate and arranged external the housing 140. In one example, the system 100 includes the optical sensor 120, wireless communication module 177, and processor 175 that are arranged within the housing 140, wherein the optical sensor 120 captures the image 112i, the processor 175 analyses the image 112i, and the wireless communication module 177 transmits (e.g., using a wireless protocol such as Bluetooth (BT) or any of 802.11 (WiFi)) the recommendation to a separate device located elsewhere within the home of the use, such as to a smartphone carried by the user 114 or a television location in a sitting room, and wherein the separate device includes the display 110 and renders the recommendations for the user 114. However, the system 100 may include any number of components arranged within or external the housing 140. As used herein the terms optical sensor 120 and camera 170 may be used interchangeably to denote an image capture system and/or device for capturing the image 112i and outputting one or more signals representative of the captured image 112i. Image 112i may be captured in still format or video (e.g., moving image) format.

As depicted in FIGS. 1A and 1B, the housing 140 of the system 100 includes optical sensor 120 and is configured for arrangement within a bathroom or other location, and includes a mirrored external surface 130. The mirrored external surface 130 is preferably planar and preferably defines a substantial portion of a broad face of the housing 140. The housing 140 preferably includes a feature, such as a mounting bracket or fastener (not shown) that enables the housing to be mounted to a wall (e.g., wall 179) or the like. The housing 140 is preferably an injection-molded plastic component, though the housing may alternatively be machined, stamped, vacuum formed, blow molded, spun, printed, or otherwise manufactured from aluminum, steel, Nylon, ABS, HDPE, or any other metal, polymer, or other suitable material.

As depicted in FIG. 1A, the optical sensor 120 of the system 100 is arranged within the housing 140 and is configured to record the image 112i including the face 112f of the user 114. The optical sensor 120 is preferably a digital color camera (e.g., camera 170). However, the optical sensor 120 may be any one or more of an RGB camera, a black and white camera, a charge-coupled device (CCD) sensor, a complimentary metal-oxide-semiconductor (CMOS) active pixel sensor, or other suitable sensor. The optical sensor 120 is preferably arranged within the housing 140 with the field of view of the optical sensor 120 extending out of the broad face of the housing 140 including the mirrored external surface 130. The optical sensor 120 is preferably adjacent the mirrored external surface 130, through the optical sensor 120 may alternatively be arranged behind the mirrored external surface 130 or in any other way on or within the housing 140.

The optical sensor 120 preferably records the image 112i of the user 114 that is a video feed including consecutive still images 102 with red 101, green 103, and blue 105 color signal components. However, the image 112i may be a still image 102, including any other additional or alternative color signal component 101, 103, 105), or be of any other form or composition. The image 112i preferably includes and is focused on the face 112f of the user 114, though the image may be of any other portion of the user 114.

The optical sensor 120 preferably records the image 112i of the user 114 automatically, i.e. without a prompt or input from the user 114 directed specifically at the system 100. In one variation of the system 100, the optical sensor 120 interfaces with a speaker or other audio sensor incorporated into the system 100, wherein an audible sound above a threshold sound level may activate the optical sensor 120. For example, the sound of a closing door, running water, or a footstep may activate the optical sensor 120. In another variation of the system 100, the optical sensor 120 interfaces with an external sensor that detects a motion or action external the system. For example, a position sensor coupled to a bathroom faucet 181 and the system 100 may activate the optical sensor 120 when the faucet 181 is opened. In another example, a pressure sensor arranged on the floor proximal a bathroom sink 180, such as in a bathmat or a bath scale (e.g., a wirelessly-enabled scale 190, such as a bathmat scale), activates the optical sensor 120 when the user 114 stands on or trips the pressure sensor. In a further variation of the system 100, the optical sensor 120 interfaces with a light sensor that detects when a light has been turned on a room, thus activating the optical sensor. In this variation, the optical sensor 120 may perform the function of the light sensor, wherein the optical sensor 120 operates in a low-power mode (e.g., does not focus, does not use a flash, operates at a minimum viable frame rate) until the room is lit, at which point the optical sensor 120 switches from the low-power setting to a setting enabling capture of a suitable image 112i of the user 114. In yet another variation of the system 100, the optical sensor 120 interfaces with a clock, timer, schedule, or calendar of the user 114. For example, for a user 114 who consistently wakes and enters the bathroom within a particular time window, the optical sensor 120 may be activated within the particular time window and deactivated outside of the particular time window. In this example, the system 100 may also learn habits of the user 114 and activate and deactivate the optical sensor 120 (e.g., to reduce power consumption) accordingly. In another example, the optical sensor 120 may interface with an alarm clock of the user 114, wherein, when the user 114 deactivates an alarm, the optical sensor 120 is activated and remains so for a predefined period of time. In a further variation of the system 100, the optical sensor interfaces 120 (e.g., via wireless module 177) with a mobile device (e.g., cellular phone) carried by the user 114, wherein the optical sensor 120 is activated when the mobile device is determined to be substantially proximal the system 100, such as via GPS, a cellular, Wi-Fi, or Bluetooth connection, near-field communications, or a RFID chip or tag indicating relative location or enabling distance- or location-related communications between the system 100 and the mobile device. However, the optical sensor 120 may interface with any other component, system, or service and may be activated or deactivated in any other way. Furthermore, the processor 175, remote server, or other component or service controlling the optical sensor 120 may implement facial recognition such that the optical sensor 120 only captures the image 112i of the user 114 (or the processor 175 or remote server only analyses the image 112i) when the user 114 is identified in the field of view of the optical sensor 120 (or within the image).

The optical sensor 120 preferably operates in any number of modes, including an ‘off’ mode, a low-power mode, an ‘activated’ mode, and a ‘record’ mode. The optical sensor 120 is preferably off or in the low-power mode when the user 114 is proximal or not detected as being proximal the system 100. As described above the optical sensor 120 preferably does not focus, does not use a flash, and/or operates at a minimum viable frame rate in the low-power mode. In the activated mode, the optical sensor 120 may be recording the image 112i or simply be armed for recordation and not recording. However, the optical sensor 120 may function in any other way.

As depicted in FIG. 1B, the system may further include processor 175 that is configured to identify the first current health indicator by analyzing the image 112i of the face 112f of the user 114. Additionally or alternatively and as described above, the system 100 may interface (e.g., via wireless module 177) with a remote server that analyzes the image 112i and extracts the first current health indicator. In this variation of the system 100, the remote server may further generate and transmit the first and/or second recommendations to the system 100 for presentation to the user 114.

The processor 175 and/or remote server preferably implements machine vision to extract at least one of the heart rate, the respiratory rate, the temperature, the posture, a facial feature, a facial muscle position, and/or facial swelling of the user from the image 112i thereof.

In one variation, the system 100 extracts the heart rate and/or the respiratory rate of the user 114 from the image 112i that is a video feed, as described in U.S. Provisional Application Ser. No. 61/641,672, filed on 2 May 2012, and titled “Method For Determining The Heart Rate Of A Subject”, already incorporated by reference herein in its entirety for all purposes.

In another variation, the system 100 implements thresholding, segmentation, blob extraction, pattern recognition, gauging, edge detection, color analysis, filtering, template matching, or any other suitable machine vision technique to identify a particular facial feature, facial muscle position, or posture of the user 114, or to estimate the magnitude of facial swelling or facial changes.

The processor 175 and/or remote server may further implement machine learning to identify any health-related metric or feature of the user 114 in the image 112i. In one variation of the system 100, the processor 175 and/or remote server implements supervised machine learning in which a set of training data of facial features, facial muscle positions, postures, and/or facial swelling is labeled with relevant health-related metrics or features. A learning procedure then preferably transforms the training data into generalized patterns to create a model that may subsequently be used to extract the health-related metric or feature from the image 112i. In another variation of the system 100, the processor 175 and/or remote server implements unsupervised machine learning (e.g., clustering) or semi-supervised machine learning in which all or at least some of the training data is not labeled, respectively. In this variation, the processor 175 and/or remote server may further implement feature extraction, principle component analysis (PCA), feature selection, or any other suitable technique to identify relevant features or metrics in and/or to prune redundant or irrelevant features from the image 112i of the user 114.

In the short-term, the processor 175 and/or remote server may associate any one or more of the health-related metrics or features with user stress. In an example implementation, any one or more of elevated user heart rate, elevated user respiratory rate, rapid body motions or head jerks, and facial wrinkles may indicate that the user 114 is currently experiencing an elevated stress level. For example, an elevated user heart rate accompanied by a furrowed brow may suggest stress, which may be distinguished from an elevated user heart rate and lowered eyelids that suggest exhaustion after exercise. Furthermore, any of the foregoing user metrics or features may be compared against threshold values or template features of other users, such as based upon the age, gender, ethnicity, demographic, location, or other characteristic of the user, to identify the elevated user stress level. Additionally or alternatively, any of the foregoing user metrics or features may be compared against historic user data to identify changes or fluctuations indicative of stress. For example, a respiratory rate soon after waking that is significantly more rapid than normal may suggest that the user is anxious or nervous for an upcoming event. In the short-term, the estimated elevated stress level of the user 114 may inform the first recommendation that is a suggestion to cope with current stressor. For example, the display 110 may render the first recommendation that is a suggestion for the user 114 to count to ten or to sit down and breathe deeply, which may reduce the heart rate and/or respiratory rate of the user 114. By sourcing additional user data, such as time, recent user location (e.g., a gym or work), a post or status on a social network, credit card or expenditure data, or a calendar, elevated user heart rate and/or respiratory rate related to stress may be distinguished from that of other factors, such as physical exertion, elation, or other positive factors.

Over the long-term, user stress trends may be generated by correlating user stress with particular identified stressors. User stress trends may then inform the second recommendation that includes a suggestion to avoid, combat, or cope with sources of stress. Additionally or alternatively, user stress may be correlated with the weight of the user 114 over time. For example, increasing incidence of identified user stress over time that occurs simultaneously with user weight gain may result in a second, long-term recommendation that illustrates a correlation between stress and weight gain for the user 114 and includes preventative suggestions to mitigate the negative effects of stress or stressors on the user 114. In this example, the second recommendation may be a short checklist of particular, simple actions shown to aid the user 114 in coping with external factors or stressors, such as to a reminder to bring a poop bag when walking the dog in the morning, to pack the next day's lunch the night before, to pack a computer power cord before leaving work, and to wash and fold laundry each Sunday. The system 100 may therefore reduce user stress by providing timely reminders of particular tasks, particularly when the user is occupied with other obligations, responsibilities, family, or work.

Current elevated user heart rate and/or respiratory rate may alternatively indicate recent user activity, such as exercise, which may be documented in a user activity journal. Over the long-term, changes to weight, stress, sleep or exhaustion level, or any other health indicator of the user 114 may be correlated with one or more user activities, as recorded in the user activity journal. Activities correlating with positive changes to user health may then be reinforced by the second recommendation. Additionally or alternatively, the user 114 may be guided away from activities correlating with negative user health changes in the second recommendation. For example, consistent exercise may be correlated with a reduced user resting heart rate of the user 114 and user weight loss, and the second recommendation presented to the user 114 every morning on the display 110 may depict this correlation (e.g., in graphical form) and suggest that the user 114 continue the current regimen. In another example, forgetting to take allergy medication at night before bed during the spring may be correlated with decreased user productivity and energy level on the following day, and the second recommendation presented to the user 114 each night during the spring may therefore include a suggestion to take an allergy medication at an appropriate time.

In the short-term, the processor 175 and/or remote server may also or alternatively associate any one or more of the health-related metrics or features with user mood. In general, user posture, facial wrinkles, and/or facial muscle position, identified in the image 112i of the user 114, may indicate a current mood or emotion of the user 114. For example, sagging eyelids and stretched skin around the lips and cheeks may correlate with amusement, a drooping jaw line and upturned eyebrows may correlate with interest, and heavy forehead wrinkles and squinting eyelids may correlate with anger. As described above, additional user data may be accessed and associated with the mood of the user 114. In the short-term, the first recommendation may include a suggestion to prolong or harness a positive mood or a suggestion to overcome a negative mood. Over the long-term, estimated user moods may be correlated with user experiences and/or external factors, and estimated user moods may thus be added to a catalogue of positive and negative user experiences and factors. This mood catalogue may then inform second recommendations that include suggestions to avoid and/or to prepare in advance for negative experiences and factors.

The processor 175 and/or remote server may also or alternatively associate any one or more of the health-related metrics or features with user sleep or exhaustion. In one variation, periorbital swelling (i.e. bags under the eyes) identified in the face 112f of the user 114 in the image 112i is associated with user exhaustion or lack of sleep. Facial swelling identified in the image 112i may be analyzed independently or in comparison with past facial swelling of the user 114 to generate an estimation of user exhaustion, sleep quality, or sleep quantity. In the long-term, user activities, responsibilities, expectations, and sleep may be prioritized and/or optimized to best ensure that the user 114 fulfills the most pressing responsibilities and obligations and completes desired activities and expectations with appropriate sleep quantity and/or quality. This optimization may then be preferably presented to the user 114 on the display 110. For example, for the user 114 who loves to cook but typically spends three hours cooking each night at the expense of eating late and sleeping less, the second recommendation may be for a recipe with less prep time such that the user 114 may eat earlier and sleep longer while still fulfilling a desire to cook. In another example, for the user 114 who typically awakes to an alarm in the middle of a REM cycle, the second recommendation may be to set an alarm earlier to avoid waking in the middle of REM sleep. In this example, all or a portion of the system 100 may be arranged adjacent a bed of the user 114 or in communication with a device adjacent the bed of the user 114, wherein the system 100 or other device measures the heart rate and/or respiratory rate of the user 114 through not contact means while the user sleeps, such as described in U.S. Provisional Application Ser. No. 61/641,672, filed on 2 May 2012, and titled “Method For Determining The Heart Rate Of A Subject”, already incorporated by reference herein in its entirety for all purposes.

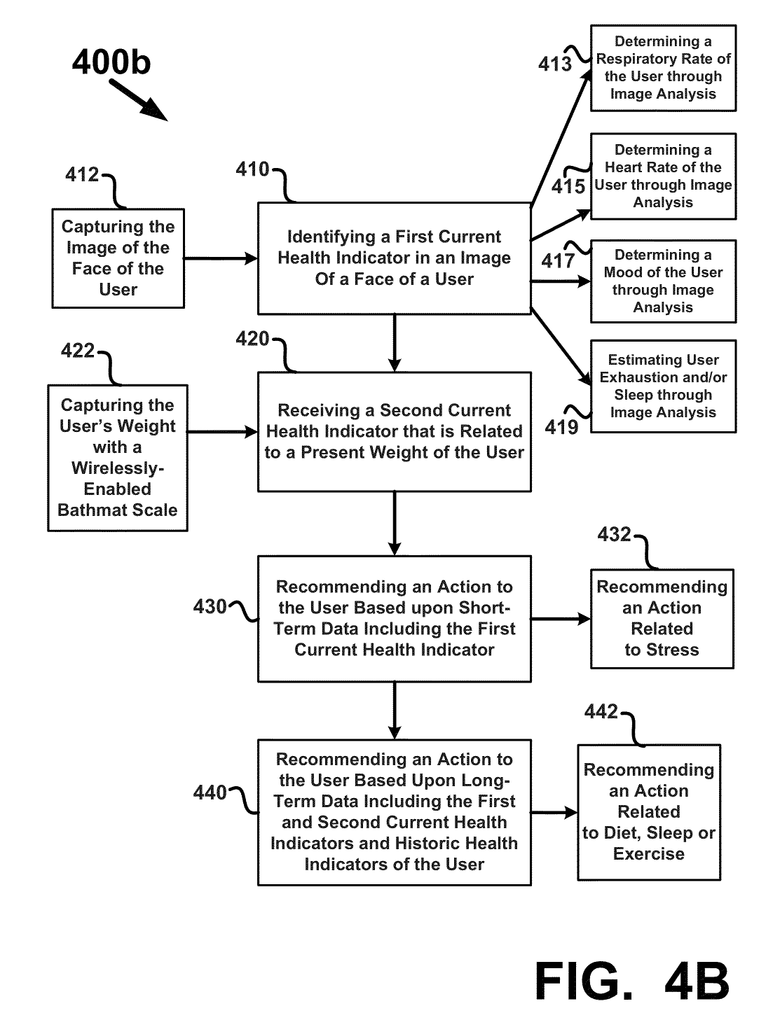

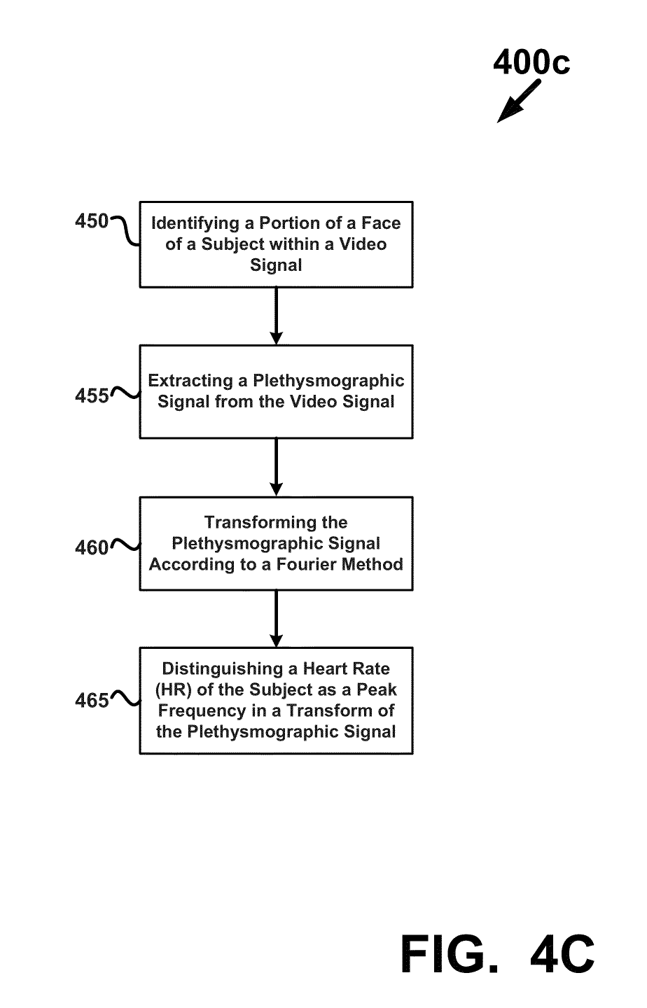

Alternatively, the system 100 may interface with a variety of devices, such as a biometric or motion sensor worn by the user 114 while sleeping or during other activities, such as a heart rate sensor or accelerometer, or any other device or sensor configured to capture user sleep data or other data for use in the methods (e.g., flow charts) described in FIGS. 4A-6. For example, while asleep, the user 114 may wear a data capable strap band, wristband, wristwatch, digital watch, or wireless activity monitoring and reporting device that monitors user biometric data including but not limited to: heart rate; respiratory rate; sleep parameters such as REM sleep, periods of deep sleep and/or light sleep; periods of being awake; and temperature, just to name a few. The biometric data may be communicated to system 100 using a wired connection (e.g., USB, Ethernet, LAN, Firewire, Thunderbolt, Lightning, etc.) or a wireless connection (e.g., BT, WiFi, NFC, RFID, etc.).

In the long term, the processor 175 and/or remote server may also or alternatively access user dietary data, such as from a user dietary profile maintained on a local device, mobile device, or remote network and consistently updated by the user 114. For example, the system 100 may access ‘The Eatery,’ a mobile dietary application accessible on a smartphone or other mobile device carried by the user 114. Dietary trends may be associated with trends in user weight, stress, and/or exercise, to generate the second recommendation that suggests changes, improvements, and/or maintenance of user diet, user stress coping mechanisms, and user exercise plan. For example, periods of high estimated user stress may be correlated with a shift in user diet toward heavily-processed foods and subsequent weight gain, and the second recommendation may therefore include suggestions to cope with or overcome stress as well as suggestions for different, healthier snacks. However, the system 100 may account for user diet in any other way in generating the first and/or second recommendations.

The processor 175 and/or remote server may also or alternatively estimate if the user 114 is or is becoming ill. For example, facial analyses of the user 114 in consecutive images 112i may show that the cheeks on face 112f of the user 114 are slowly sinking, which is correlated with user illness. The system 100 may subsequently generate a recommendation that is to see a doctor, to eat certain foods to boost user immune system, to stay home from work or school to recover, or local sickness trends to suggest a particular illness and correlated risk or severity level. However, other use biometric data, such as heart rate or respiratory rate, may also or alternatively indicate if the user 114 is or is becoming sick, and the system 100 may generate any other suitable illness-related recommendation for the user 114.

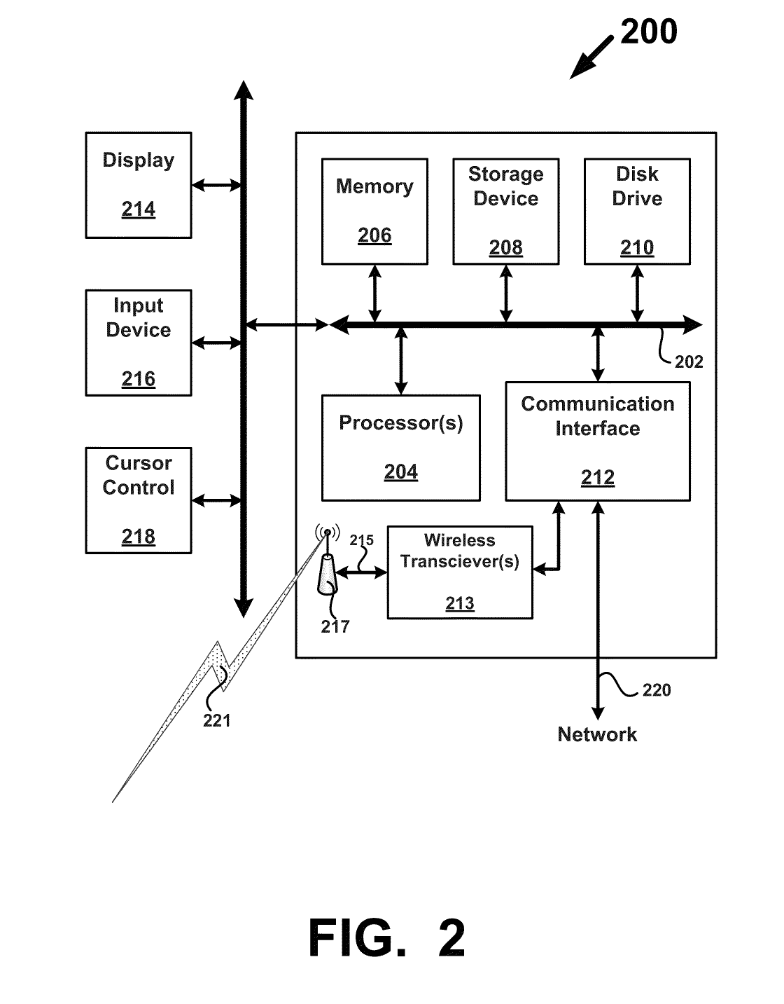

FIG. 2 depicts an exemplary computer system 200 suitable for use in the systems, methods, and apparatus described herein for estimating body fat in a user. In some examples, computer system 200 may be used to implement computer programs, applications, configurations, methods, processes, or other software to perform the above-described techniques. Computer system 200 includes a bus 202 or other communication mechanism for communicating information, which interconnects subsystems and devices, such as one or more processors 204, system memory 206 (e.g., RAM, SRAM, DRAM, Flash), storage device 208 (e.g., Flash, ROM), disk drive 210 (e.g., magnetic, optical, solid state), communication interface 212 (e.g., modem, Ethernet, WiFi), display 214 (e.g., CRT, LCD, touch screen), input device 216 (e.g., keyboard, stylus), and cursor control 218 (e.g., mouse, trackball, stylus). Some of the elements depicted in computer system 200 may be optional, such as elements 214-218, for example and computer system 200 need not include all of the elements depicted.

According to some examples, computer system 200 performs specific operations by processor 204 executing one or more sequences of one or more instructions stored in system memory 206. Such instructions may be read into system memory 206 from another non-transitory computer readable medium, such as storage device 208 or disk drive 210 (e.g., a HD or SSD). In some examples, circuitry may be used in place of or in combination with software instructions for implementation. The term “non-transitory computer readable medium” refers to any tangible medium that participates in providing instructions to processor 204 for execution. Such a medium may take many forms, including but not limited to, non-volatile media and volatile media. Non-volatile media includes, for example, optical, magnetic, or solid state disks, such as disk drive 210. Volatile media includes dynamic memory, such as system memory 206. Common forms of non-transitory computer readable media includes, for example, floppy disk, flexible disk, hard disk, SSD, magnetic tape, any other magnetic medium, CD-ROM, DVD-ROM, Blu-Ray ROM, USB thumb drive, SD Card, any other optical medium, punch cards, paper tape, any other physical medium with patterns of holes, RAM, PROM, EPROM, FLASH-EPROM, any other memory chip or cartridge, or any other medium from which a computer may read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 202 for transmitting a computer data signal. In some examples, execution of the sequences of instructions may be performed by a single computer system 200. According to some examples, two or more computer systems 200 coupled by communication link 220 (e.g., LAN, Ethernet, PSTN, or wireless network) may perform the sequence of instructions in coordination with one another. Computer system 200 may transmit and receive messages, data, and instructions, including programs, (i.e., application code), through communication link 220 and communication interface 212. Received program code may be executed by processor 204 as it is received, and/or stored in disk drive 210, or other non-volatile storage for later execution. Computer system 200 may optionally include a wireless transceiver 213 in communication with the communication interface 212 and coupled 215 with an antenna 217 for receiving and generating RF signals 221, such as from a WiFi network, BT radio, or other wireless network and/or wireless devices, for example. Examples of wireless devices include but are not limited to: a data capable strap band, wristband, wristwatch, digital watch, or wireless activity monitoring and reporting device; a smartphone; cellular phone; tablet; tablet computer; pad device (e.g., an iPad); touch screen device; touch screen computer; laptop computer; personal computer; server; personal digital assistant (PDA); portable gaming device; a mobile electronic device; and a wireless media device, just to name a few. Computer system 200 in part or whole may be used to implement one or more components of system 100 of FIGS. 1A-1C. For example, processor 175, wireless module 177, display 110, and optical sensor 120 may be implemented using one or more elements of computer system 200. Computer system 200 in part or whole may be used to implement a remote server or other compute engine in communication with system 100 of FIGS. 1A-1C.

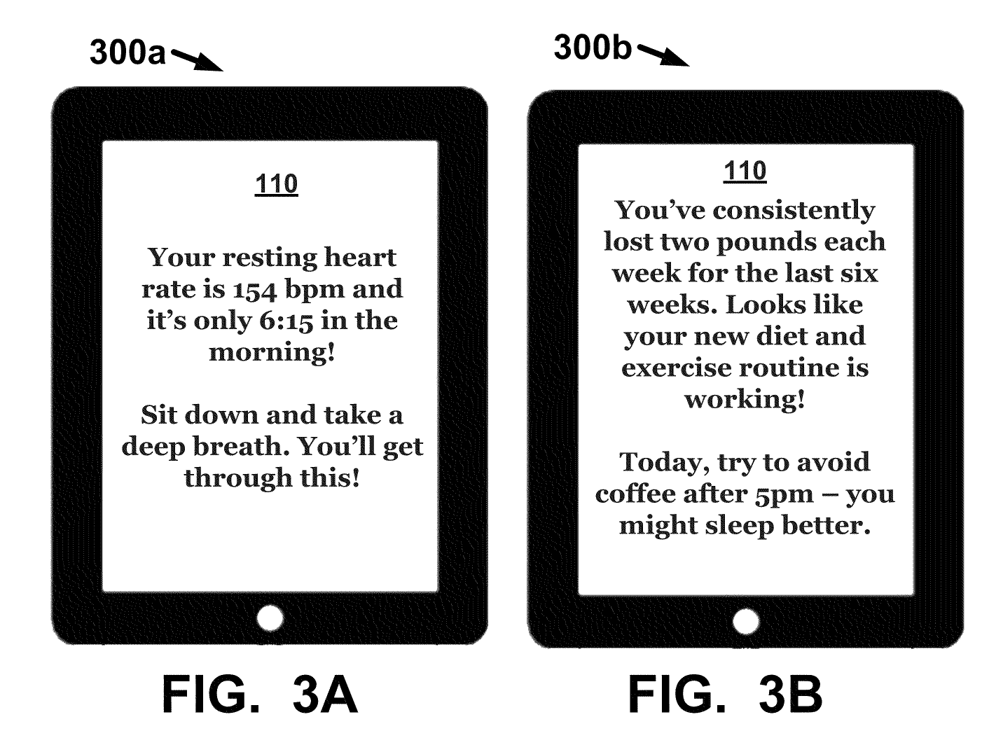

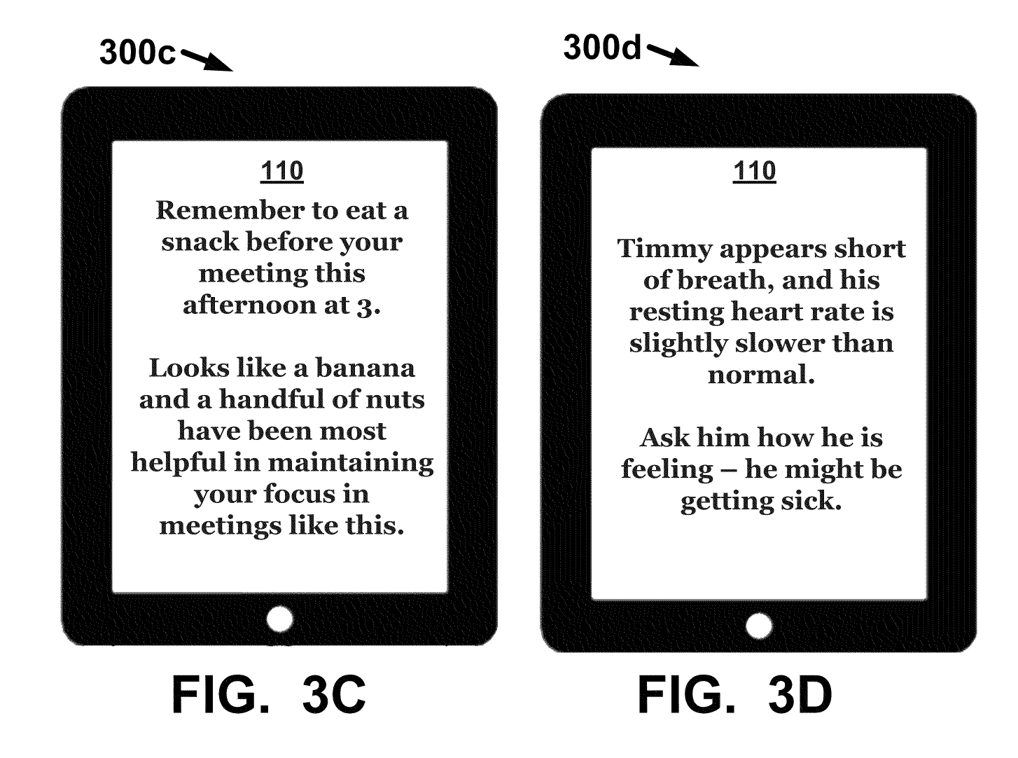

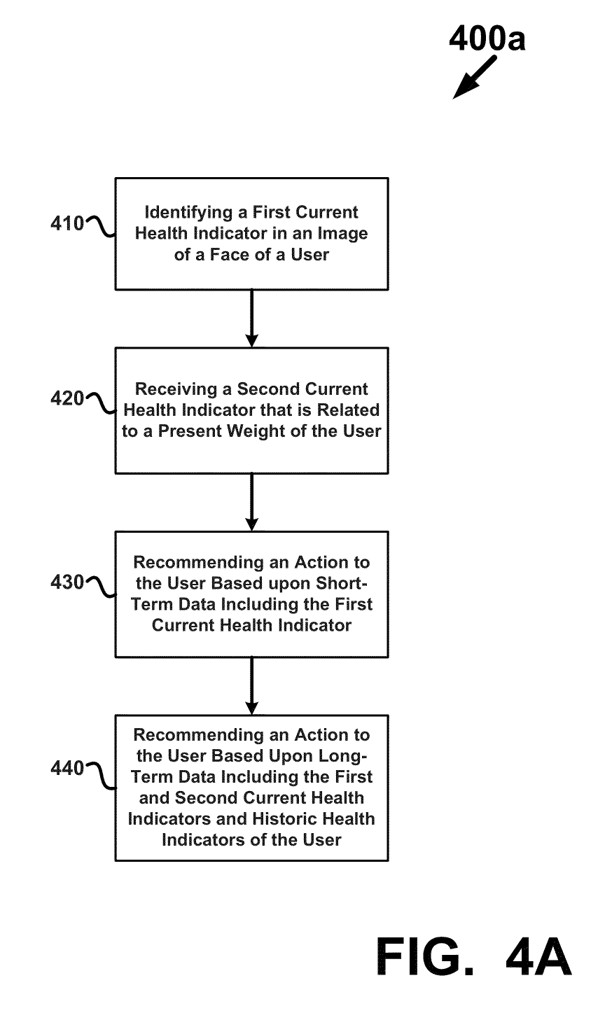

The system 100 may additionally or alternatively provide a recommendation that is an answer or probably solution to an automatically- or user-selected question, as depicted in FIGS. 3A-3D. The question may be any of: “are my kids getting sick;” “am I brushing my teeth long enough;” “when should I go to bed to look most rested in the morning;” “how long am I sleeping a night;” “is my heart getting more fit;” “is my face getting fatter;” “how does stress affect my weight;” “is my workout getting me closer to my goals;” “are my health goals still appropriate;” “what affects my sleeps;” “are the bags under my eyes getting darker;” “is there anything strange going on with my heart;” “how stressed am I;” “how does my calendar look today;” “did I remember to take my medications;” or “am I eating better this week than last?” However, the system 100 may answer or provide a solution to any other question relevant to the user 114.

As depicted in FIG. 1A, the display 110 of the system 100 is arranged within the housing 140 and adjacent the mirrored surface 130. The display 110 is further configured to selectively render the first recommendation and the second recommendation for the user 114. The display 110 may be any of a liquid crystal, plasma, segment, LED, OLED, or e-paper display, or any other suitable type of display. The display 110 is preferably arranged behind the mirrored external surface 130 and is preferably configured to transmit light through the mirrored external surface 130 to present the recommendations to the user 114. However, the display 110 may be arranged beside the mirrored external surface 130 or in another other way on or within the housing 140. Alternatively, the display 110 may be arranged external the housing 140. For example, the display 110 may be arranged within a second housing that is separated from the housing 140 and that contains the optical sensor 120. In another example, the display 110 may be a physically coextensive with a cellular phone, tablet, mobile electronic device, laptop or desktop computer, digital watch, vehicle display, television, gaming console, PDA, digital music player, or any other suitable electronic device carried by, user 114 by, or interacting with the user 114.

Attention is now directed to FIG. 1C where a functional block diagram 199 depicts one example of an implementation of a physiological characteristic determinator 150. Diagram 199 depicts physiological characteristic determinator 150 coupled with a light capture device 104, which also may be an image capture device (e.g., 120, 170), such as a digital camera (e.g., video camera). As shown, physiological characteristic determinator 150 includes an orientation monitor 152, a surface detector 154, a feature filter 156, a physiological signal extractor 158, and a physiological signal generator 160. Surface detector 154 is configured to detect one or more surfaces associated with an organism, such as a person (e.g., user 114). As shown, surface detector 154 may use, for example, pattern recognition or machine vision, as described herein, to identify one or more portions of a face of the organism (e.g., face 112f). As shown, surface detector 154 detects a forehead portion 111a and one or more cheek portions 111b. For example, cheek portions 111b may comprise an approximately symmetrical set of features on face 112f, that is cheek portions 112b are approximately symmetrical about a center line 112c. Surface detector 154 may be configured to detect at least one set of symmetrical facial features (e.g., cheek portions 111b) and optionally at least one other facial feature which may or may not be symmetrical and/or present as a set. Feature filter 156 is configured to identify features other than those associated with the one or more surfaces to filter data associated with pixels representing the features. For example, feature filter 156 may identify feature 113, such as the eyes, nose, and mouth to filter out related data associated with pixels representing the features 113. Thus, physiological characteristic determinator 150 processes certain face portions and “locks onto” those portions for analysis (e.g., portions of face 112f).

Orientation monitor 152 is configured to monitor an orientation 112 of the face (e.g., face 112f) of the organism (e.g., user 114), and to detect a change in orientation in which at least one face portion is absent. For example, the organism may turn its head away, thereby removing a cheek portion 111b from image capture device 104. For example, in FIG. 1C, the organism may turn its head to the side 112s thereby removing a front of the face 112f from view of the image capture device. In response, physiological characteristic determinator 150 may compensate for the absence of cheek portion 111b, for example, by enlarging the surface areas of the face portions, by amplifying or weighting pixel values and/or light component magnitudes differently, or by increasing the resolution in which to process pixel data, just to name a few examples.

Physiological signal extractor 158 is configured to extract one or more signals including physiological information from subsets of light components captured by light capture device 104. For example, each subset of light components may be associated with one or more frequencies and/or wavelengths of light. According to some embodiments, physiological signal extractor 158 identifies a first subset of frequencies (e.g., a range of frequencies, including a single frequency) constituting green visible light, a second subset of frequencies constituting red visible light, and a third subset of frequencies constituting blue visible light. According to other embodiments, physiological signal extractor 158 identifies a first subset of wavelengths (e.g., a range of wavelengths, including a single wavelength) constituting green visible light, a second subset of wavelengths constituting red visible light, and a third subset of wavelengths constituting blue visible light. Other frequencies and wavelengths are possible, including those outside visible spectrum. As shown, a signal analyzer 159 of physiological signal extractor 158 is configured to analyze the pixel values or other color-related signal values 117a (e.g., green light), 117b (e.g., red light), and 117c (e.g., green light). For example, signal analyzer 159 may identify a time-domain component associated with a change in blood volume associated with the one or more surfaces of the organism. In some embodiments, physiological signal extractor 158 is configured to aggregate or average one or more AC signals from one or more pixels over one or more sets of pixels. Signal analyzer 159 may be configured to extracting a physiological characteristic based on, for example, a time-domain component based on, for example, using Independent Component Analysis (“ICA”) and/or a Fourier Transform (e.g., a FFT).

Physiological data signal generator 160 may be configured to generate a physiological data signal 115 representing one or more physiological characteristics. Examples of such physiological characteristics include a heart rate pulse wave rate, a heart rate variability (“HRV”), and a respiration rate, among others, in a non-invasive manner.

According to some embodiments, physiological characteristic determinator 150 may be coupled to a motion sensor, 104 such as an accelerometer or any other like device, to use motion data from the motion sensor to determine a subset of pixels in a set of pixels based on a predicted distance calculated from the motion data. For example, consider that pixel or group of pixels 171 are being analyzed in association with a face portion. Upon detecting a motion (of either the organism or the image capture device, or both) in which such motion with move face portion out from pixel or group of pixels 171. Surface detector 154 may be configured to, for example, detect motion of a portions of the face in a set of pixels 117c, which affects a subset of pixels 171 including a face portion from the one or more portions of the face. Surface detector 154 predicts a distance in which the face portion moves from the subset of pixels 171 and determines a next subset of pixels 173 in the set of pixels 117c based on the predicted distance. Then, reflected light associated with the next subset of pixels 173 may be used for analysis.

In some embodiments, physiological characteristic determinator 150 may be coupled to a light sensor 107 (e.g., 104, 120, 170). Signal analyzer 159 may be configured to compensate for a value of light received from the light sensor 107 that indicates a non-conforming amount of light. For example, consider that the light source generating the light is a fluorescent light source that, for instance, provides for less than desirable amount of, for example, green light. Signal analyzer 159 may compensate, for example, by weighting values associated with either the green light (e.g., either higher) or other values associated with other subsets of light components, such as red and blue light (e.g., weight the blue and red light to decrease influence of red and blue light). Other compensation techniques are possible.

In some embodiments, physiological characteristic determinator 150, and a device in which it is disposed, may be in communication (e.g., wired or wirelessly) with a mobile device, such as a mobile phone or computing device. In some cases, such a mobile device, or any networked computing device (not shown) in communication with physiological characteristic determinator 150, may provide at least some of the structures and/or functions of any of the features described herein. As depicted in FIG. 1C and subsequent figures (or preceding figures), the structures and/or functions of any of the above-described features may be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1C (or any figure) may represent one or more algorithms. Or, at least one of the elements may represent a portion of logic including a portion of hardware configured to provide constituent structures and/or functionalities.

For example, physiological characteristic determinator 150 and any of its one or more components, such as an orientation monitor 152, a surface detector 154, a feature filter 156, a physiological signal extractor 158, and a physiological signal generator 160, may be implemented in one or more computing devices (i.e., any video-producing device, such as mobile phone, a wearable computing device, such as UP® or a variant thereof), or any other mobile computing device, such as a wearable device or mobile phone (whether worn or carried), that include one or more processors configured to execute one or more algorithms in memory. Thus, at least some of the elements in FIG. 1C (or any figure) may represent one or more algorithms. Or, at least one of the elements may represent a portion of logic including a portion of hardware configured to provide constituent structures and/or functionalities. These may be varied and are not limited to the examples or descriptions provided.

As hardware and/or firmware, the above-described structures and techniques may be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological characteristic determinator 150 and any of its one or more components, such as an orientation monitor 152, a surface detector 154, a feature filter 156, a physiological signal extractor 158, and a physiological signal generator 160, may be implemented in one or more circuits. Thus, at least one of the elements in FIG. 1C (or any figure) may represent one or more components of hardware. Or, at least one of the elements may represent a portion of logic including a portion of circuit configured to provide constituent structures and/or functionalities.

According to some embodiments, the term “circuit” may refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit may include a system of electronic components and logic components (e.g., logic configured to execute instructions, such that a group of executable instructions of an algorithm, for example, and, thus, is a component of a circuit). According to some embodiments, the term “module” may refer, for example, to an algorithm or a portion thereof, and/or logic implemented in either hardware circuitry or software, or a combination thereof (i.e., a module may be implemented as a circuit). In some embodiments, algorithms and/or the memory in which the algorithms are stored are “components” of a circuit. Thus, the term “circuit” may also refer, for example, to a system of components, including algorithms. These may be varied and are not limited to the examples or descriptions provided.

As depicted in FIGS. 3A-3D, in addition to rendering the recommendations for the user 114, the display 110 may also depict other relevant data, such as the weather forecast, a user calendar, upcoming appointments or meetings, incoming messages, emails, or phone calls, family health status, updates of friends or connections on a social network, a shopping list, upcoming flight or travel information, news, blog posts, or movie or television clips. However, the display 110 may function in any other way and render and other suitable content. In FIG. 3A, display 110 renders 300a information (heart rate and time) as well as a recommendation to user 114 as to how to lower the heart rate. In FIG. 3B, display 110 renders 300b encouragement regarding weight loss (e.g., as measured and logged from wirelessly-enabled bathmat scale 190 or other type of wirelessly-enabled scale or weight measurement device) and a recommendation as to how to get better sleep. In FIG. 3C, display 110 renders 300c a reminder and a recommendation regarding diet. In FIG. 3D, display 110 renders 300d information on biometric data regarding the health status of a user (e.g., a child) and a recommendation to query the user to see how their feeling. The foregoing are non-limiting examples of information that may be presented on display 110 as an output of system 100. The information displayed on display 110 may be based in part or whole on the first current health indicator, second current health indicator, or both and/or the recommending an action to user 114 based on short-term data, recommending an action to user 114 based on long-term data, or both.

As depicted in FIG. 1B, one variation of the system further includes a wireless communication module 177 that receives 193 user-related data from an external device. The wireless communication module 177 preferably wirelessly receives 193 weight (or mass) measurements of the user 114, such as from a wirelessly-enabled bath scale 190. As described above, the wireless communication module 177 may additionally or alternatively gather user-related data from a biometric or action sensor worn by the user 114, a remote server, a mobile device carried by the user 114, an external sensor, or any other suitable external device, network, or server. The wireless communication module 177 may further transmits 178 the first and/or second recommendations to a device worn or carried by the user 114, a remote server, an external display, or any other suitable external device, network, or server.

As depicted in FIG. 1B, one variation of the system further includes a bathmat scale 190 configured to determine the weight of the user 114 when the user stands 192 (depicted by dashed arrow) on the bathmat scale 190, wherein the bathmat scale 190 is further configured to transmit (e.g., wirelessly using wireless unit 191) the weight of the user 114 to the processor 175 and/or remote server to inform the second current health indicator. The bathmat scale 190 is preferably and absorbent pad including a pressure sensor, though the bathmat scale 190 may alternatively be a pressure sensor configured to be arranged under a separate bathmat. However, the bathmat scale 190 may be of any other form, include any other sensor, and function in any other way. Furthermore, the system 100 may exclude the bathmat scale 190 and/or exclude communications with a bath scale 190, wherein the user 114 manually enters user weight, or wherein the system 100 gleans user weight data from alternative sources, such as a user health record. Bathmat scale 190 may optionally include a wireless unit 191 configured to wirelessly communicate 193 with processor 175 via wireless module 177 and/or with a remote server, the weight of the user 114.

In one variation, the system 100 may further function as a communication portal between the user 114 and a second user (not shown). Through the system 100, the user 114 may access the second user to discuss health-related matters, such as stress, a dietary or exercise plan, or sleep patterns. Additionally or alternatively, the user 114 may access the system 100 to prepare for a party or outing remotely with the second user, wherein the system 100 transmits audio and/or visual signals of the user 114 and second user between the second user and the user 114. However, the system 100 may operate in any other way and perform any other function.

Moving now to FIG. 4A, a method 400a for monitoring the health of a user 114 includes: identifying a first current health indicator in an image 112i of a face 112f of the user 114 at a stage 410; receiving a second current health indicator related to a present weight of the user 114 at a stage 420 (e.g., from wirelessly-enabled bathmat scale 190); recommending an action to the user 114 based upon short-term data including the first current health indicator (e.g., from stage 410) at a stage 430; and recommending an action to the user 114 based upon long-term data including the first and second current health indicators (e.g., from stages 410 and 420) and historic health indicators of the user 114 at a stage 440. Stages 410-440 may be implemented using hardware (e.g., circuitry), software (e.g., executable code fixed in a non-transitory computer readable medium), or both. System 100 may implement some or all of the stages 410-440, or another system (e.g., computer system 200 of FIG. 2) external to system 100 may implement some or all of the stages 410-440.

As depicted in FIGS. 4A and 4B, the methods 400a and/or 400b may be implemented as an application executing on the system 100 described above, wherein methods 400a and/or 400b enable the functions of the system 100 described above. Alternatively, methods 400a and/or 400b may be implemented as an applet or application executing in whole or in part on the remote server described above or as a website accessible by the system 100 (e.g., via wireless module 177), though methods 400a and/or 400b may be implemented in any other way.

Turning now to FIG. 4B, a method 400b includes a plurality of additional stages that may optionally be performed with respect to stages 410-440 of FIG. 4A. In connection with stage 410, a stage 412 may comprise capturing an image 112i of a face 112f of the user 114 to provide the image for the stage 410. The image 112i may be captured using the above described optical sensor 120, camera 170, or image capture device 104, for example. A stage 422 may comprise capturing the weight of user 114 using the wirelessly enabled bathmat scale 190, or some other weight capture device, to provide the present weight of the user 114 for the stage 420. In other examples, the weight of user 114 may be input manually (e.g., using a smartphone, tablet, or other wired/wirelessly enabled device). The weight or user 114 may be obtained from a database or other source, such as the Internet, Cloud, web page, remote server, etc.

The stage 410 may comprise one or more adjunct stages denoted as stages 413-419. The stage 410 may include determining a respiratory rate of the user 114 by performing image analysis of the image 112i as depicted at a stage 413. The stage 410 may include determining a heart rate of the user 114 by performing image analysis of the image 112i as depicted at a stage 415. The stage 410 may include determining a mood of the user 114 by performing image analysis of the image 112i as depicted at a stage 417. The stage 410 may include estimating user exhaustion and/or user sleep of the user 114 by performing image analysis of the image 112i as depicted at a stage 419.

The stages 430 and/or 440 may comprise one or more adjunct stages denoted as stages 432 and 442, respectively. Stage 430 may comprise recommending, to the user 114, an action related to stress of the user 114 as denoted by a stage 432. Analysis of the image 112i may be used to determine that the user 114 is under stress. Stage 442 may comprise recommending an action related to diet, sleep, or exercise to user 114. Analysis of the image 112i may be used to determine which recommendations related to diet, sleep, or exercise to make to user 114.

Attention is now directed to FIG. 4C, where a method 400c for determining a physiological characteristic is depicted. Method 400c provides for the determination of a physiological characteristic, such as the heart rate (HR) of a subject (e.g., user 114) or organism. As depicted, method 400c includes: identifying a portion of the face of the subject within a video signal at a stage 450; extracting or otherwise isolating a plethysmographic signal in the video signal through independent component analysis at a stage 455; transforming the plethysmographic signal according to a Fourier method (e.g., a Fourier Transform, FFT) at a stage 460; and identifying a heart rate (HR) of the subject as a peak frequency in the transform (e.g., Fourier transform or other transform) of the plethysmographic signal at a stage 465.

Method 400c may function to determine the HR of the subject through non-contact means, specifically by identifying fluctuations in the amount of blood in a portion of the body of the subject (e.g., face 112f), as captured in a video signal (e.g., from 120, 170, 104), through component analysis of the video signal and isolation of a frequency peak in a Fourier transform of the video signal. Method 400c may be implemented as an application or applet executing on an electronic device incorporating a camera, such as a cellular phone, smartphone, tablet, laptop computer, or desktop computer, wherein stages of the method 400c are completed in part or in whole by the electronic device. Stages of method 400c may additionally or alternatively be implemented by a remote server or network in communication with the electronic device. Alternatively, the method 400c may be implemented as a service that is remotely accessible and that serves to determine the HR of a subject in an uploaded, linked, or live-feed video signal, though the method 400c may be implemented in any other way. In the foregoing or any other variation, the video signal and pixel data and values generated therefrom are preferably a live feed from the camera in the electronic device, though the video signal may be preexisting, such as a video signal recorded previously with the camera, a video signal sent to the electronic device, or a video signal downloaded from a remote server, network, or website. Furthermore, method 400c may also include calculating the heart rate variability (HRV) of the subject and/or calculating the respiratory rate (RR) of the subject, or any other physiological characteristic, such as a pulse wave rate, a Meyer wave, etc.

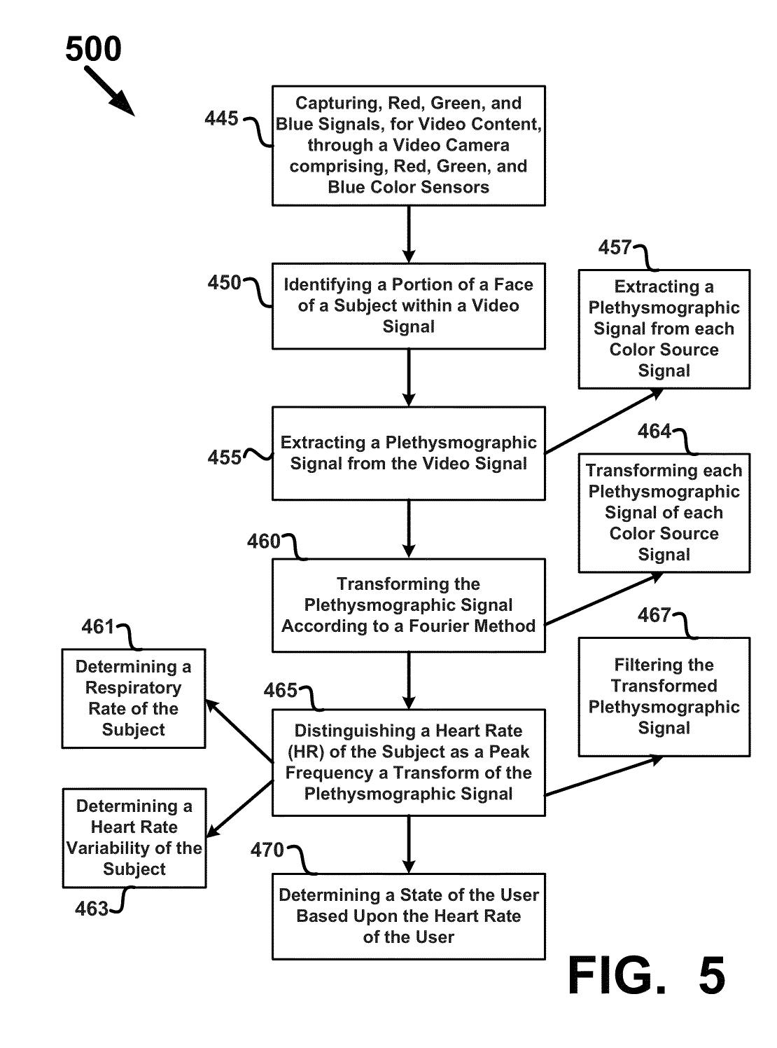

In the example depicted in FIG. 4C, a variation of the method 400c is depicted in FIG. 5 where a method 500 includes a stage 445, for capturing red, green, and blue signals, for video content, through a video camera including red, green, and blue color sensors. Stage 445 may therefore function to capture data necessary to determine the HR of the subject (e.g., face 112f of user 114) without contact. The camera is preferably a digital camera (or optical sensor) arranged within an electronic device carried or commonly used by the subject, such as a smartphone, tablet, laptop or desktop computer, computer monitor, television, or gaming console. Device 100 and image capture devices 120, 170, and 104 may be user for the video camera that includes red, green, and blue color sensors.

The video camera preferably operates at a known frame rate, such as fifteen or thirty frames per second, or other suitable frame rate, such that a time-domain component is associated with the video signal. The video camera may also preferably incorporates a plurality of color sensors, including distinct red, blue, and green color sensors, each of which generates a distinct red, blue, and green source signal, respectively. The color source signal from each color sensor is preferably in the form of an image for each frame recorded by the video camera. Each color source signal from each frame may thus be fed into a postprocessor implementing other Blocks of the method 400c and/or 500 to determine the HR, HRV, and/or RR of the subject. In some embodiments, a light capture device may be other than a camera or video camera, but may include any type of light (of any wavelength) receiving and/or detecting sensor.

As depicted in FIG. 4C and FIG. 5, stage 450 of methods 400c and 500, recites identifying a portion of the face of the subject within the video signal. Blood swelling in the face, particularly in the cheeks and forehead, occurs substantially synchronously with heartbeats. A plethysmographic signal may thus be extracted from images of a face captured and identified in a video feed. Stage 450 may preferably identify the face of the subject because faces are not typically covered by garments or hair, which would otherwise obscure the plethysmographic signal. However, stage 450 may additionally or alternatively include identifying any other portion of the body of the subject, in the video signal, from which the plethysmographic signal may be extracted.

Stage 450 may preferably implement machine vision to identify the face in the video signal. In one variation, stage 450 may use edge detection and template matching to isolate the face in the video signal. In another variation, stage 450 may implement pattern recognition and machine learning to determine the presence and position of the face 112f in the video signal. This variation may preferably incorporate supervised machine learning, wherein stage 450 accesses a set of training data that includes template images properly labeled as including or not including a face. A learning procedure may then transform the training data into generalized patterns to create a model that may subsequently be used to identify a face in video signals. However, in this variation, stage 450 may alternatively implement unsupervised learning (e.g., clustering) or semi-supervised learning in which at least some of the training data has not been labeled. In this variation, stage 450 may further implement feature extraction, principle component analysis (PCA), feature selection, or any other suitable technique to prune redundant or irrelevant features from the video signal. However, stage 450 may implement edge detection, gauging, clustering, pattern recognition, template matching, feature extraction, principle component analysis (PCA), feature selection, thresholding, positioning, or color analysis in any other way, or use any other type of machine learning or machine vision to identify the face 112f of the subject (e.g., user 114) in the video signal.

In stage 450, each frame of the video feed, and preferably each frame of each color source signal of the video feed, may be cropped of all image data excluding the face 112f or a specific portion of the face 112f of the subject (e.g., user 114). By removing all information in the video signal that is irrelevant to the plethysmographic signal, the amount of time required to calculate subject HR may be reduced.

As depicted in FIG. 4C, stage 455 of method 400c recites extracting a plethysmographic signal from the video signal. In the variation of the method 400c in which the video signal includes red, green, and blue source signals, stage 455 may preferably implement independent component analysis to identify a time-domain oscillating (AC) component, in at least one of the color source signals, that includes the plethysmographic signal attributed to blood volume changes in or under the skin of the portion of the face identified in stage 450. Stage 455 may preferably further isolate the AC component from a DC component of each source signal, wherein the DC component may be attributed to bulk absorption of the skin rather than blood swelling associated with a heartbeat. The plethysmographic signal isolated in the stage 455 therefore may define a time-domain AC signal of a portion of a face of the subject shown in a video signal. However, multiple color source-dependent plethysmographic signal(s) may be extracted in stage 455, wherein each plethysmographic signal defines a time-domain AC signal of a portion of a face of the subject identified in a particular color source signal in the video feed. However, each plethysmographic signal may be extracted from the video signal in any other way in stage 455.

The plethysmographic signal that is extracted from the video signal in stage 455 may preferably be an aggregate or averaged AC signal from a plurality of pixels associated with a portion of the face 112f of the subject identified in the video signal, such as either or both cheeks 111b or the forehead 111a of the subject. By aggregating or averaging an AC signal from a plurality of pixels, errors and outliers in the plethysmographic signal may be minimized. Furthermore, multiple plethysmographic signals may be extracted in stage 455 for each of various regions of the face 112f, such as each cheek 111b and the forehead 111a of the subject, as shown in FIG. 1C. However, stage 455 may function in any other way and each plethysmographic signal may be extracted from the video signal according to any other method.

As depicted in FIG. 4C, stage 460 of method 400c recites transforming the plethysmographic signal according to a Fourier transform. Stage 460 may preferably convert the plethysmographic time-domain AC signal to a frequency-domain plot. In a variation of the method 400c in which multiple plethysmographic signals are extracted (e.g., as in stage 457 of method 500), such as a plethysmographic signal for each of several color source signals and/or for each of several portions of the face 112f of the user 114, the stage 460 may preferably include transforming each of the plethysmographic signals separately to create a time-domain waveform of the AC component of each plethysmographic signal (e.g., as in stage 464 of method 500). Stage 460 may additionally or alternatively include transforming the plethysmographic signal according to, for example, a Fast Fourier Transform (FFT) method, though stage 460 may function in any other way (e.g., using any other similar transform) and according to any other method.

As depicted in FIG. 4C, stage 465 of method 400c recites distinguishing the HR of the subject as a peak frequency in the transform of the plethysmographic signal. Because a human heart may beat at a rate in range from about 40 beats per minute (e.g., highly-conditioned adult athlete at rest) to about 200 beats for minute (e.g., highly-active child), stage 465 may preferably function by isolating a peak frequency within a range of about 0.65 Hz to about 4 Hz, converting the peak frequency to a beats per minute value, and associating the beats per minute value with the HR of the subject.

In one variation of the method 400c as depicted in method 500 of FIG. 5, isolation of the peak frequency is limited to the anticipated frequency range that corresponds with an anticipated or possible HR range of the subject. In another variation of the method 400c, the frequency-domain waveform of the stage 460 is filtered at a stage 467 of FIG. 5 to remove waveform data outside of the range of about 0.65 Hz to about 4 Hz. For example, at the stage 467, the plethysmographic signal may be fed through a bandpass filter configured to remove or attenuate portions of the plethysmographic signal outside of the predefined frequency range. Generally, by filtering the frequency-domain waveform of stage 460, repeated variations in the video signal, such as color, brightness, or motion, falling outside of the range of anticipated HR values of the subject may be stripped from the plethysmographic signal and/or ignored. For example, alternating current (AC) power systems in the United States operate at approximately 60 Hz, which results in oscillations of AC lighting systems on the order of 60 Hz. Though this oscillation may be captured in the video signal and transformed in stage 460, this oscillation falls outside of the bounds of anticipated or possible HR values of the subject and may thus be filtered out or ignored without negatively impacting the calculated subject HR, at least in some embodiments.

In the variation of the method 400c as depicted in method 500 of FIG. 5, in which multiple plethysmographic signals are transformed in the stage 464, stage 464 may include isolating the peak frequency in each of the transformed (e.g., frequency-domain) plethysmographic signals. The multiple peak frequencies may then be compared in the stage 465, such as by removing outliers and averaging the remaining peak frequencies to calculate the HR of the subject. Particular color source signals may be more efficient or more accurate for estimating subject HR via the method 400c and/or method 500, and the particular transformed plethysmographic signals may be given greater weight when averaged with less accurate plethysmographic signals.

Alternatively, in the variation of the method 400c in which multiple plethysmographic signals are transformed in the stage 460 and/or stage 464, stage 465 may include combining the multiple transformed plethysmographic signals into a composite transformed plethysmographic signal, wherein a peak frequency is isolated in the composite transformed plethysmographic signal to estimate the HR of the subject. However, stage 465 may function in any other way and implement any other mechanisms.

In a variation of the method 400c as depicted in method 500 in FIG. 5, stage 465 may further include a stage 463 for determining a heart rate variability (HRV) of the subject through analysis of the transformed plethysmographic signal of stage 460. HRV may be associated with power spectral density, wherein a low frequency power component of the power spectral density waveform or the video signal or a color source signal thereof may reflect sympathetic and parasympathetic influences. Furthermore, the high frequency powers component of the power spectral density waveform may reflect parasympathetic influences. Therefore, in this variation, stage 465 may preferably isolate sympathetic and parasympathetic influences on the heart through power spectral density analysis of the transformed plethysmographic signal to determine HRV of the subject.

In a variation of the method 400c as depicted in method 500 in FIG. 5, the stage 465 may further include a stage 461 for determining a respiratory rate (RR) of the subject through analysis of the transformed plethysmographic signal of the stage 460. In this variation, stage 465 may preferably derive the RR of the subject through the high frequency powers component of the power spectral density, which is associated with respiration of the subject.

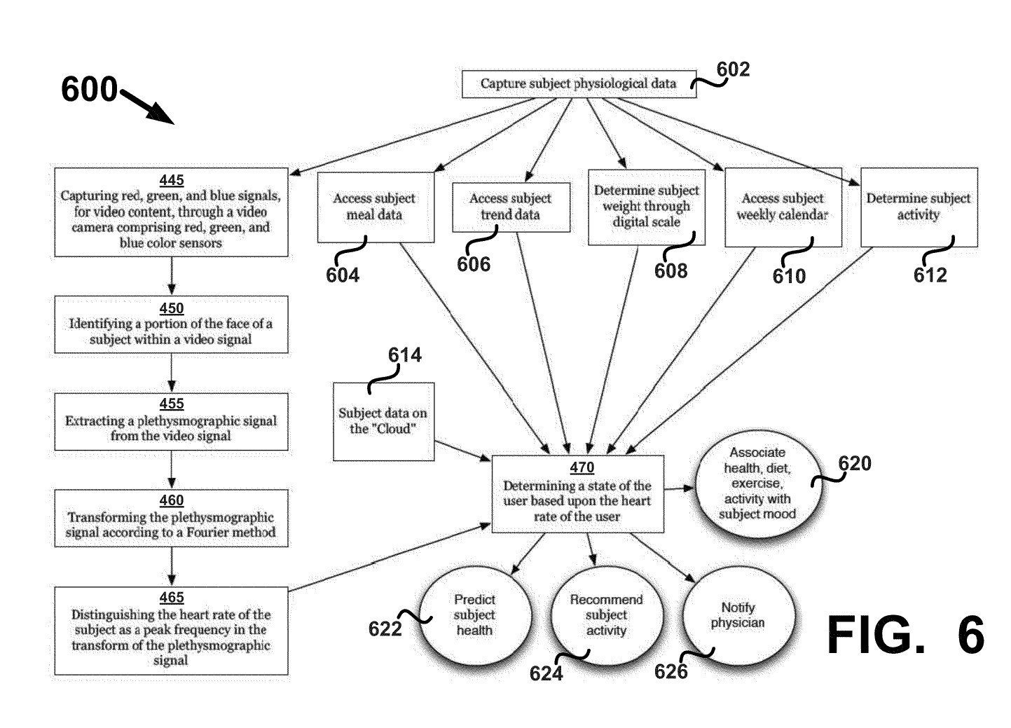

As depicted in FIGS. 5-6, methods 500 and 600 may further include a stage 470, which recites determining a state of the user based upon the HR thereof. In stage 470, the HR, HRV, and/or RR of the subject are preferably augmented with an additional subject input, data from another sensor, data from an external network, data from a related service, or any other data or input. Stage 470 therefore may preferably provide additional functionality applicable to a particular field, application, or environment of the subject, such as described below.

FIG. 6 depicts an example of a varied flow, according to some embodiments. As shown in method 600, method 400c of FIG. 4C is a component of method 600. At a stage 602, physiological characteristic data of an organism (e.g., user 114) may be captured and applied to further processes, such as computer programs or algorithms, to perform one or more of the following. At a stage 604, nutrition and meal data may be accessed for application with the physiological data. At a stage 606, trend data and/or historic data may be used along with physiological data to determine whether any of actions at stages 620 to 626 ought to be taken. Other information may be determined from a stage 608 at which an organism's weight (i.e., fat amounts) is obtained (e.g., from wirelessly-enabled bathmat scale 190). At a stage 610, a subject's calendar data is accessed and an activity in which the subject is engaged is determined at a stage 612 to determine whether any of actions at stages 620 to 626 ought to be taken.

By enabling a mobile device, such as a smartphone or tablet, to implement one or more of the methods 400c, 500, or 600, the subject may access any of the aforementioned calculations and generate other fitness-based metrics substantially on the fly and without sophisticated equipment. The methods 400c, 500, or 600, as applied to exercise, are preferably provided through a fitness application (“fitness app”) executing on the mobile device, wherein the app stores subject fitness metrics, plots subject progress, recommends activities or exercise routines, and/or provides encouragement to the subject, such as through a digital fitness coach. The fitness app may also incorporate other functions, such as monitoring or receiving inputs pertaining to food consumption or determining subject activity based upon GPS or accelerometer data.

Referring back to FIG. 6, in another variation of the stage 470, the method 600, 400c, or 500 may be applied to health. Hereinafter, method 600 will be described although the description may apply to method 400c, method 500, or both. Stage 470 may be configured to estimate a health factor of the subject. In one example implementation, the method 600 is implemented in a plurality of electronic devices, such as a smartphone, tablet, and laptop computer that communicate with each other to track the HR, HRV, and/or RR of the subject over time at the stage 606 and without excessive equipment or affirmative action by the subject. For example, each instance of an activity at the stage 612 in which the subject picks up his smartphone to make a call, check email, reply to a text message, read an article or e-book, or play Angry Birds, the smartphone may implement the method 600 to calculate the HR, HRV, and/or RR of the subject. Furthermore, while the subject works in front of a computer during the day or relaxes in front of a television at night, the similar data may be obtained and aggregated into a personal health file of the subject. This data is preferably pushed, from each aforementioned device, to a remote server or network that stores, organizes, maintains, and/or evaluates the data. This data may then be made accessible to the subject, a physician or other medical staff, an insurance company, a teacher, an advisor, an employer, or another health-based app. Alternatively, this data may be added to previous data that is stored locally on the smartphone, on a local hard drive coupled to a wireless router, on a server at a health insurance company, at a server at a hospital, or on any other device at any other location.

HR, HRV, and RR, which may correlate with the health, wellness, and/or fitness of the subject, may thus be tracked over time at the stage 606 and substantially in the background, thus increasing the amount of health-related data captured for a particular subject while decreasing the amount of positive action necessary to capture health-related data on the part of the subject, a medical professional, or other individual. Through the method 600, or methods 400c or 500, health-related information may be recorded substantially automatically during normal, everyday actions already performed by a large subset of the population.

With such large amounts of HR, HRV, and/or RR data for the subject, health risks for the subject may be estimated at the stage 622. In particular, trends in HR, HRV, and/or RR, such as at various times or during or after certain activities, may be determined at the stage 612. In this variation, additional data falling outside of an expected value or trend may trigger warnings or recommendations for the subject. In a first example, if the subject is middle-aged and has a HR that remains substantially low and at the same rate throughout the week, but the subject engages occasionally in strenuous physical activity, the subject may be warned of increased risk of heart attack and encouraged to engage is light physical activity more frequently at the stage 624. In a second example, if the HR of the subject is typically 65 bpm within five minutes of getting out of bed, but on a particular morning the HR of the subject does not reach 65 bpm until thirty minutes after rise, the subject may be warned of the likelihood of pending illness, which may automatically trigger confirmation a doctor visit at the stage 626 or generation a list of foods that may boost the immune system of the subject. Trends may also show progress of the subject, such as improved HR recovery throughout the course of a training or exercise regimen.

In this variation, method 600 may also be used to correlate the effect of various inputs on the health, mood, emotion, and/or focus of the subject. In a first example, the subject may engage an app on his smartphone (e.g., The Eatery by Massive Health) to record a meal, snack, or drink. While inputting such data, a camera on the smartphone may capture the HR, HRV, and/or RR of the subject such that the meal, snack, or drink may be associated with measured physiological data. Overtime, this data may correlate certain foods correlate with certain feelings, mental or physical states, energy levels, or workflow at the stage 620. In a second example, the subject may input an activity, such as by “checking in” (e.g., through a Foursquare app on a smartphone) to a location associated with a particular product or service. When shopping, watching a sporting event, drinking at a pub with friends, seeing a movie, or engaging in any other activity, the subject may engage his smartphone for any number of tasks, such as making a phone call or reading an email. When engaged by the user, the smartphone may also capture subject HR and then tag the activity, location, and/or individuals proximal the user with measured physiological data. Trend data at the stage 606 may then be used to make recommendations to the subject, such as a recommendation to avoid a bar or certain individuals because physiological data indicates greater anxiety or stress when proximal the bar or the certain individuals. Alternatively, an elevated HR of the subject while performing a certain activity may indicate engagement in and/or enjoyment of the activity, and the subject may subsequently be encouraged to join friends who are currently performing the activity. Generally, at the stage 610, social alerts may be presented to the subject and may be controlled (and scheduled), at least in part, by the health effect of the activity on the subject.

In another example implementation, the method 600 may measure the HR of the subject who is a fetus. For example, the microphone integral with a smartphone may be held over a woman's abdomen to record the heart beats of the mother and the child. Simultaneously, the camera of the smartphone may be used to determine the HR of the mother via the method 600, wherein the HR of the woman may then be removed from the combined mother-fetus heart beats to distinguish heart beats and the HR of the fetus alone. This functionality may be provided through software (e.g., a “baby heart beat app”) operating on a standard smartphone rather than through specialized. Furthermore, a mother may use such an application at any time to capture the heartbeat of the fetus, rather than waiting to visit a hospital. This functionality may be useful in monitoring the health of the fetus, wherein quantitative data pertaining to the fetus may be obtained at any time, thus permitting potential complications to be caught early and reducing risk to the fetus and/or the mother. Fetus HR data may also be cumulative and assembled into trends, such as described above.

Generally, the method 600 may be used to test for certain heart or health conditions without substantial or specialized equipment. For example, a victim of a recent heart attack may use nothing more than a smartphone with integral camera to check for heart arrhythmia. In another example, the subject may test for risk of cardiac arrest based upon HRV. Recommendations may also be made to the subject, such as based upon trend data, to reduce subject risk of heart attack. However, the method 600 may be used in any other way to achieve any other desired function.

Further, method 600 may be applied as a daily routine assistant. Block S450 may be configured to include generating a suggestion to improve the physical, mental, or emotional health of the subject substantially in real time. In one example implementation, the method 600 is applied to food, exercise, and/or caffeine reminders. For example, if the subject HR has fallen below a threshold, the subject may be encouraged to eat. Based upon trends, past subject data, subject location, subject diet, or subject likes and dislikes, the type or content of a meal may also be suggested to the subject. Also, if the subject HR is trending downward, such as following a meal, a recommendation for coffee may be provided to the subject. A coffee shop may also be suggested, such as based upon proximity to the subject or if a friend is currently at the coffee shop. Furthermore, a certain coffee or other consumable may also be suggested, such as based upon subject diet, subject preferences, or third-party recommendations, such as sourced from Yelp. The method 600 may thus function to provide suggestions to maintain an energy level and/or a caffeine level of the subject. The method 600 may also provide “deep breath” reminders. For example, if the subject is composing an email during a period of elevated HR, the subject may be reminded to calm down and return to the email after a period of reflection. For example, strong language in an email may corroborate an estimated need for the subject to break from a task. Any of these recommendations may be provided through pop-up notifications on a smartphone, tablet, computer, or other electronic device, through an alarm, by adjusting a digital calendar, or by any other communication means or through any other device.

In another example implementation, the method 600 may be used to track sleep patterns. For example, a smartphone or tablet placed on a nightstand and pointed at the subject may capture subject HR and RR throughout the night. This data may be used to determine sleep state, such as to wake up the subject at an ideal time (e.g., outside of REM sleep). This data may alternatively be used to diagnose sleep apnea or other sleep disorders. Sleep patterns may also be correlated with other factors, such as HR before bed, stress level throughout the day (as indicated by elevated HR over a long period of time), dietary habits (as indicated through a food app or changes in subject HR or RR at key times throughout the day), subject weight or weight loss, daily activities, or any other factor or physiological metric. Recommendations for the subject may thus be made to improve the health, wellness, and fitness of the subject. For example, if the method 600 determines that the subject sleeps better, such as with fewer interruptions or less snoring, on days in which the subject engages in light to moderate exercise, the method 600 may include a suggestion that the subject forego an extended bike ride on the weekend (as noted in a calendar) in exchange for shorter rides during the week. However, any other sleep-associated recommendation may be presented to the subject.

The method 600 may also be implemented through an electronic device configured to communicate with external sensors to provide daily routine assistance. For example, the electronic device may include a camera and a processor integrated into a bathroom vanity, wherein the HR, HRV, and RR of the subject is captured while the subject brushes his teeth, combs his hair, etc. A bathmat (e.g., 190) in the bathroom may include a pressure sensor configured to capture at the stage 608 the weight of the subject, which may be transmitted to the electronic device. The weight, hygiene, and other action and physiological factors may thus all be captured in the background while a subject prepares for and/or ends a typical day. However, the method 600 may function independently or in conjunction with any other method, device, or sensor to assist the subject in a daily routine.

Other applications of the stage 470 of FIG. 6 are possible. For example, the method 600 may be implemented in other applications, wherein the stage 470 determines any other state of the subject. In a one example, the method 600 may be used to calculate the HR of a dog, cat, or other pet. Animal HR may be correlated with a mood, need, or interest of the animal, and a pet owner may thus implement the method 600 to further interpret animal communications. In this example, the method 600 is preferably implemented through a “dog translator app” executing on a smartphone or other common electronic device such that the pet owner may access the HR of the animal without additional equipment. In this example, a user may engage the dog translator app to quantitatively gauge the response of a pet to certain words, such as “walk,” “run,” “hungry,” “thirsty,” “park,” or “car,” wherein a change in pet HR greater than a certain threshold may be indicative of a current desire of the pet. The inner ear, nose, lips, or other substantially hairless portions of the body of the animal may be analyzed to determine the HR of the animal in the event that blood volume fluctuations within the cheeks and forehead of the animal are substantially obscured by hair or fur.

In another example, the method 600 may be used to determine mood, interest chemistry, etc. of one or more actors in a movie or television show. A user may point an electronic device implementing the method 600 at a television to obtain an estimate of the HR of the actor(s) displayed therein. This may provide further insight into the character of the actor(s) and allow the user to understand the actor on a new, more personal level. However, the method 600 may be used in any other way to provide any other functionality.

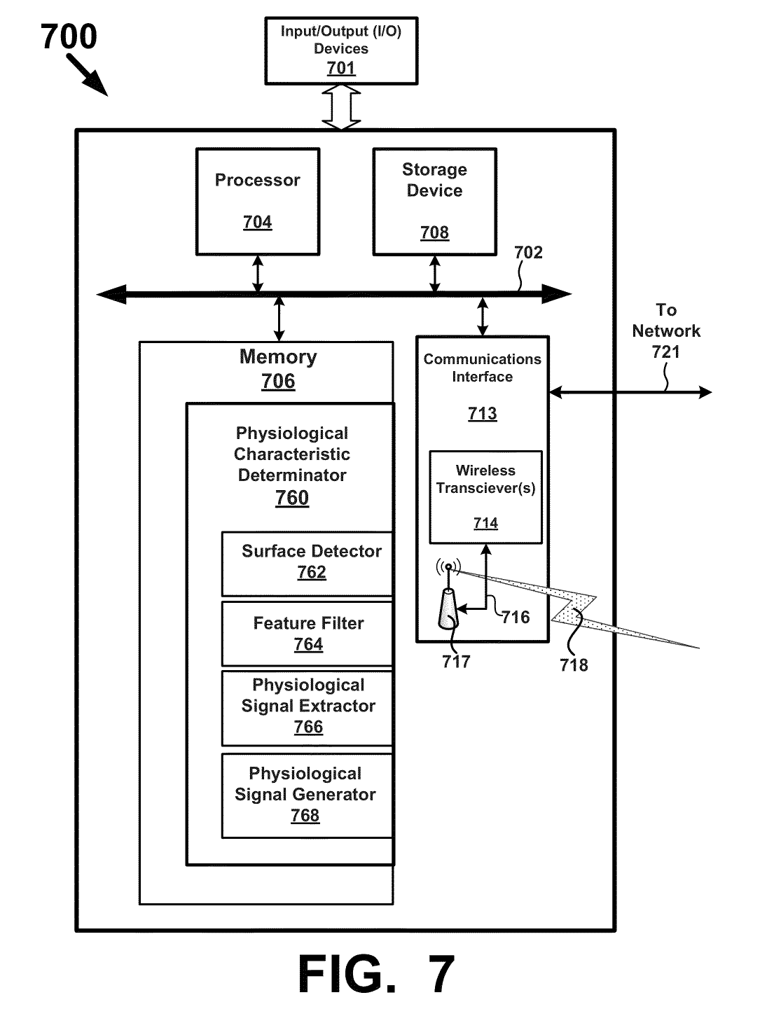

FIG. 7 depicts another exemplary computing platform disposed in a computing device in accordance with various embodiments. In some examples, computing platform 700 may be used to implement computer programs, applications, methods, processes, algorithms, or other software to perform the above-described techniques. Computing platform 700 includes a bus 702 or other communication mechanism for communicating information, which interconnects subsystems and devices, such as processor 704, system memory 706 (e.g., RAM, Flash, DRAM, SRAM, etc.), storage device 708 (e.g., ROM, Flash, etc.), a communication interface 713 (e.g., an Ethernet or wireless controller, a Bluetooth controller, etc.) to facilitate communications via a port on communication link 721 to communicate, for example, with a computing device, including mobile computing and/or communication devices with processors. Optionally, communication interface 713 may include one or more wireless transceivers 714 electrically coupled 716 with and antenna 717 and configured to send and receive wireless transmissions 718. Processor 704 may be implemented with one or more central processing units (“CPUs”), such as those manufactured by Intel® Corporation, or one or more virtual processors, as well as any combination of CPUs, DSPs, and virtual processors. Computing platform 700 exchanges data representing inputs and outputs via input-and-output devices 701, including, but not limited to, keyboards, mice, stylus, audio inputs (e.g., speech-to-text devices), an image sensor, a camera, user interfaces, displays, monitors, cursors, touch-sensitive displays, LCD or LED displays, and other I/O-related devices.

According to some examples, computing platform 700 performs specific operations by processor 704 executing one or more sequences of one or more instructions stored in system memory 706 (e.g., executable instructions embodied in a non-transitory computer readable medium), and computing platform 700 may be implemented in a client-server arrangement, peer-to-peer arrangement, or as any mobile computing device, including smart phones and the like. Such instructions or data may be read into system memory 706 from another computer readable medium, such as storage device 708. In some examples, hard-wired circuitry may be used in place of or in combination with software instructions for implementation. Instructions may be embedded in software or firmware. The term “non-transitory computer readable medium” refers to any tangible medium that participates in providing instructions to processor 704 for execution. Such a medium may take many forms, including but not limited to, non-volatile media and volatile media. Non-volatile media includes, for example, optical or magnetic disks and the like. Volatile media includes dynamic memory, such as system memory 706.

Common forms of non-transitory computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer may read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 702 for transmitting a computer data signal.

In some examples, execution of the sequences of instructions may be performed by computing platform 700. According to some examples, computing platform 700 may be coupled by communication link 721 (e.g., a wired network, such as LAN, PSTN, or any wireless network) to any other processor to perform the sequence of instructions in coordination with (or asynchronous to) one another. Computing platform 700 may transmit and receive messages, data, and instructions, including program code (e.g., application code) through communication link 721 and communication interface 713. Received program code may be executed by processor 704 as it is received, and/or stored in memory 706 or other non-volatile storage for later execution.

In the example depicted in FIG. 7, system memory 706 may include various modules that include executable instructions to implement functionalities described herein. In the example depicted, system memory 706 includes a Physiological Characteristic Determinator 760 configured to implement the above-identified functionalities. Physiological Characteristic Determinator 760 may include a surface detector 762, a feature filter 764, a physiological signal extractor 766, and a physiological signal generator 768, each may be configured to provide one or more functions described herein.

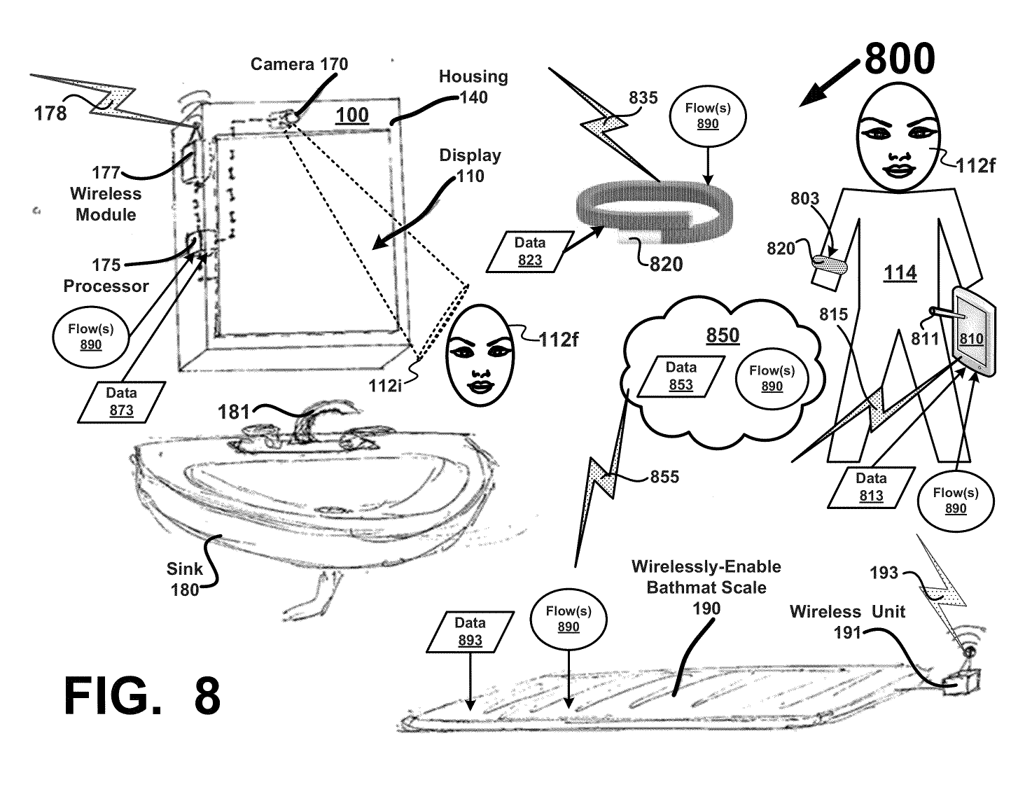

Referring now to FIG. 8 where one example of a system 800 that includes one or more wireless resources for determining the health of a user is depicted. System 800 may comprise one or more wireless resources denoted as 100, 190, 810, 820, and 850. All, or a subset of the wireless resources may be in wireless communication (178, 193, 815, 835, 855) with one another. Resource 850 may be the Cloud, Internet, server, the exemplary computer system 200 of FIG. 2, a web site, a web page, laptop, PC, or other compute engine and/or data storage system that may be accessed wirelessly by other wireless resources in system 800, in connection with one or more of the methods 400a-400c, 500, and 600 as depicted and described in reference to FIGS. 4A-6. One or more of the methods 400a-400c, 500, or 600 may be embodied in a non-transitory computer readable medium denoted generally as flows 890 in FIG. 8. Flows 890 may reside in whole or in part in one or more of the wireless resources 100, 190, 810, 820, and 850.

One or more of data 813, 823, 853, 873, and 893 may comprise data for determining the health of a user including but not limited to: biometric data; weight data; activity data; recommended action data; first and/or second current health indicator data; historic health indicator data; short term data; long term data; user weight data; image capture data from face 112f; user sleep data; user exhaustion data; user mood data; user heart rate data; heart rate variability data; user respiratory rate data; Fourier method data; data related to the plethysmographic signal; red, green, and blue image data; user meal data; trend data; user calendar data; user activity data; user diet data; user exercise data; user health data; data for transforms; and data for filters, just to name a few. Data 813, 823, 853, 873, and 893 may reside in whole or in part in one or more of the wireless resources 100, 190, 810, 820, and 850.

Data and/or flows used by system 100 may reside in a single wireless resource or in multiple wireless resources. The following are non-limiting examples of interaction scenarios between the wireless resources depicted in FIG. 8. In a first example, wireless resource 820 comprises a wearable user device such as a wear a data capable strap band, wristband, wristwatch, digital watch, or wireless activity monitoring and reporting device. In the example depicted, user 114 wears the wireless resource 820 approximately positioned at a wrist 803 on an arm of user 114. At least some of the data 823 needed for flows 890 resides in data storage within wireless resource 820. System 100 wirelessly (178, 835) accesses the data it needs from a data storage unit of wireless resource 820. Data 823 may comprise any data required by flows 890. As one example, user 114 may step 192 on scale 190 to take a weight measurement that is wirelessly (193, 835) communicated to the wireless resource 820. User 114 may take several of the weight measurements which are accumulated and logged as part of data 823. Wireless resource 820 may include one or more sensors or other systems which sense biometric data from user 114, such as heart rate, respiratory data, sleep activity, exercise activity, diet activity, work activity, sports activity, calorie intake, and calories burned, galvanic skin response, alarm setting, calendar information, and body temperature information, just to name a few. System 100 may wirelessly access 178 (e.g., via handshakes or other wireless protocols) some or all of data 823 as needed. Data 873 of system 100 may be replaced, supplanted, amended, or otherwise altered by whatever portions of data 823 are accessed by system 100. System 100 may use some or all of data (873, 823). Moreover, system 100 may use some or all of any of the other data (853, 813, 893) available to system 100 in a manner similar to that described above for data (873, 823). User 114 may cause data 823 to be manually or automatically read or written to an appropriate data storage system of resource 820, 100, or any other wireless resources. For example, user 114 standing 192 on resource 190 may automatically cause resources 820 and 190 to wirelessly link with each other, and data comprising the measured weight of user 114 is automatically wirelessly transmitted 193 to resource 820. On the other hand, user 114 may enter data comprising diet information on resource 810 (e.g., using stylus 811 or a finger to a touch screen) where the diet information is stored as data 813 and that data may be manually wirelessly communicated 815 to any of the resources, including resource 820, 100, or both. Resource 820 may gather data using its various systems and sensors while user 114 is asleep. The sleep data may then be automatically wirelessly transmitted 835 to resource 100.

Some or all of the data from wireless resources (100, 190, 810, 820) may be wirelessly transmitted 855 to resource 850 which may serve as a central access point for data. System 100 may wirelessly access the data it requires from resource 850. Data 853 from resource 850 may be wirelessly 855 transmitted to any of the other wireless resources as needed. In some examples, data 853 or a portion thereof, comprises one or more of the data 813, 823, 873, or 893. Although not depicted, a wireless network such as a WiFi network, wireless router, cellular network, or WiMAX network may be used to wirelessly connect one or more of the wireless resources with one another.

One or more of the wireless resources depicted in FIG. 8 may include one or more processors or the like for executing one or more of the flows 890 as described above in reference to FIGS. 4A-6. Although processor 175 of resource 100 may handle all of the processing of flows 890, in other examples, some or all of the processing of flows 890 is external to the system 100 and may be handled by another one or more of the wireless resources. Therefore, a copy of algorithms, executable instructions, programs, executable code, or the like required to implement flows 890 may reside in a data storage system of one or more of the wireless resources.

As one example, resource 810 may process data 813 using flows 890 and wirelessly communicate 815 results, recommendations, actions, and the like to resource 100 for presentation on display 110. As another example, resource 850 may include processing hardware (e.g., a server) to process data 853 using flows 890 and wirelessly communicate 815 results, recommendations, actions, and the like to resource 100 for presentation on display 110. System 100 may image 112i the face 112f of user 114, and then some or all of the image data (e.g., red 101, green 103, and blue 105 components) may be wirelessly transmitted 178 to another resource, such as 810 or 850 for processing and the results of the processing may be wirelessly transmitted back to system 100 where additional processing may occur and results presented on display 110 or on another resource, such as a display of resource 810. As depicted in FIG. 8, bathmat 190 may also include data 893, flows 890, or both and may include a processor and any other systems required to handle data 893 and/or flows 890 and to wirelessly communicate 193 with the other wireless resources.

The systems, apparatus and methods of the foregoing examples may be embodied and/or implemented at least in part as a machine configured to receive a non-transitory computer-readable medium storing computer-readable instructions. The instructions may be executed by computer-executable components preferably integrated with the application, server, network, website, web browser, hardware/firmware/software elements of a user computer or electronic device, or any suitable combination thereof. Other systems and methods of the embodiment may be embodied and/or implemented at least in part as a machine configured to receive a non-transitory computer-readable medium storing computer-readable instructions. The instructions are preferably executed by computer-executable components preferably integrated by computer-executable components preferably integrated with apparatuses and networks of the type described above. The non-transitory computer-readable medium may be stored on any suitable computer readable media such as RAMs, ROMs, Flash memory, EEPROMs, optical devices (CD, DVD or Blu-Ray), hard drives (HD), solid state drives (SSD), floppy drives, or any suitable device. The computer-executable component may preferably be a processor but any suitable dedicated hardware device may (alternatively or additionally) execute the instructions.

As a person skilled in the art will recognize from the previous detailed description and from the drawing FIGS. and claims set forth below, modifications and changes may be made to the embodiments of the present application without departing from the scope of this present application as defined in the following claims.

Although the foregoing examples have been described in some detail for purposes of clarity of understanding, the above-described inventive techniques are not limited to the details provided. There are many alternative ways of implementing the above-described techniques or the present application. The disclosed examples are illustrative and not restrictive.

What is claimed is:

1. A method for monitoring health, comprising:
identifying a first current health indicator in an image of facial features;
receiving a wireless signal comprised of a second current health indicator that is related to weight;
recommending a first action based upon short-term data that includes the first current health indicator; and
recommending a second action based upon long-term data that includes the first and second current health indicators and a historic health indicator.

2. The method of claim 1, wherein the image includes at least one set of symmetrical facial features and at least one non-symmetrical facial feature.

3. The method of claim 1, wherein the identifying comprises capturing the image of the facial features using an image capture device.

4. The method of claim 3, wherein the image capture device captures at least three different images of the facial features, and the at least three different images comprise a red wavelength image, a green wavelength image, and a blue wavelength image.

5. The method of claim 1, wherein the wireless signal is transmitted by a wirelessly-enabled scale configured to wirelessly transmit a signal indicative of weight.

6. The method of claim 1, wherein the identifying includes analyzing the image to determine one or more health indicators selected from the group consisting of determining a heart rate, determining a respiratory rate, and determining a mood.

7. The method of claim 1, wherein recommending the first action comprises recommending an action related to stress.

8. The method of claim 1, wherein recommending the second action comprises recommending an action related to a selected one or more of diet, sleep, or exercise.

9. The method of claim 1, wherein the identifying further comprises:
identifying a portion of the facial features of a subject within a video signal;
extracting a plethysmographic signal from the video signal;
transforming the plethysmographic signal using a Fourier method; and
distinguishing a heart rate of the subject as a peak frequency in a transform of the plethysmographic signal.

10. The method of claim 1 and further comprising:
displaying the first action on a display of a wirelessly enabled device; and
displaying the second action on the display.

11. A wirelessly-enabled system for monitoring health, comprising:
a processor;
a data storage system;
an image capture device;
a wireless module,
a display;
the data storage system, the image capture device, the wireless module, and the display are electrically coupled with the processor; and
a mirrored external surface positioned adjacent to the display and configured to optically transmit information displayed on the display and to optically reflect light from light sources other than the display.

12. The wirelessly-enabled system of claim 11 and further comprising:
a housing that includes the processor, the data storage system, the image capture device, the wireless module, the display, and the mirrored external surface.

13. The wirelessly-enabled system of claim 12, wherein the housing is configured to be mounted to a surface.

14. The wirelessly-enabled system of claim 11 and further comprising:
executable instructions disposed in a non-transitory computer readable medium included in the data storage system and configured to cause the processor to:
identify a first current health indicator in an image of facial features captured by the image capture device;
receive a wireless signal using the wireless module, the wireless signal comprised of a second current health indicator that is related to weight;
recommend a first action based upon short-term data that includes the first current health indicator, the first action is displayed on the display; and
recommend a second action based upon long-term data that includes the first and second current health indicators and a historic health indicator, the second action is displayed on the display.

15. The wirelessly-enabled system of claim 14, wherein the executable instructions include a physiological characteristic Determinator.

16. The wirelessly-enabled system of claim 14, wherein the wireless signal that is received by the wireless module is transmitted by a wirelessly-enabled scale.

17. The wirelessly-enabled system of claim 11 and further comprising:
a wirelessly-enabled scale in wireless communication with the wireless module and configured to wirelessly transmit a signal that is indicative of weight.

18. The wirelessly-enabled system of claim 11, wherein the image capture device is configured to capture at least three different images of facial features, and the at least three different images comprise a red wavelength image, a green wavelength image, and a blue wavelength image.

19. The wirelessly-enabled system of claim 11 and further comprising:
a wireless user device in wireless communication with the wireless module, the wireless user device comprises a device selected from the group consisting of a data capable strap band, a wristband, a wristwatch, a digital watch, and a wireless activity monitoring and reporting device.

20. A non-transitory computer readable medium including executable instructions for monitoring health, comprising:
instructions for identifying a first current health indicator in an image of facial features;
instructions for receiving a wireless signal comprised of a second current health indicator that is related to weight;
instructions for recommending a first action based upon short-term data that includes the first current health indicator; and
instructions for recommending a second action based upon long-term data that includes the first and second current health indicators and a historic health indicator.
